(19) United States
(12) Patent Application Publication
(10) Pub. No.: US 2015/0105772 A1
(43) Pub. Date: Apr. 16, 2015

(54) DEVICES AND METHODS FOR NERVE MODULATION

(71) Applicant: BOSTON SCIENTIFIC SCIMED, INC., MAPLE GROVE, MN (US)

(72) Inventors: JASON P. HILL, BROOKLYN PARK, MN (US); MARTIN R. WILLARD, BURNSVILLE, MN (US); JOEL N. GROFF, DELANO, MN (US); MARK L. JENSON, GREENFIELD, MN (US); SUSAN M. SHOEMAKER, ELK RIVER, MN (US); JOSEPH M. THIELEN, BUFFALO, MN (US)

(73) Assignee: BOSTON SCIENTIFIC SCIMED, INC., MAPLE GROVE, MN (US)

(21) Appl. No.: 14/512,020

(22) Filed: Oct. 10, 2014

Related U.S. Application Data
(60) Provisional application No. 61/890,687, filed on Oct. 14, 2013.
(60) Provisional application No. 61/890,711, filed on Oct. 14, 2013.

(51) Int. Cl. A61B 18/14

(52) U.S. Cl. CPC A61B 18/14; A61B 2018/00434
USPC 606/41; 427/2.1

(57) ABSTRACT

Systems for nerve and tissue modulation are disclosed. An illustrative system may include an intravascular nerve modulation system including a catheter shaft, an expandable basket and one or more energy delivery regions. The one or more energy delivery regions may be defined by regions of the basket free from an insulating material.

### CROSS-REFERENCE TO RELATED APPLICATIONS

This application claims priority under 35 U.S.C. §119 to U.S. Provisional Application Ser. No. 61/890,687, filed Oct. 14, 2013 and to U.S. Provisional Application Ser. No. 61/890,711, filed Oct. 14, 2013, the entireties of which are incorporated herein by reference.

### FIELD

The invention generally pertains to percutaneous and intravascular devices for nerve modulation and/or ablation.

### BACKGROUND

Certain treatments involve, and in some cases require, the temporary or permanent interruption or modification of select nerve function. One example treatment is renal nerve ablation, which can be used to treat conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many body tissues, such as nerves, including renal nerves, brain tissue, cardiac tissue and the tissue of other body organs, are in close proximity to blood vessels and/or other body cavities. This proximity enables the tissues to be accessed percutaneously or intravascularly through walls of the blood vessels. In some instances, it may be desirable to ablate perivascular nerves using a radio frequency (RF) electrode. In other instances, the perivascular nerves may be ablated by other techniques, including procedures that apply thermal, ultrasonic, laser, microwave, and/or other related energy sources to the vessel wall.

It may be beneficial to provide apparatuses and methods including, but not limited to, renal nerve modulation systems as well as methods of use and manufacture thereof, that provide reliable multi-point ablation.

### SUMMARY

The present disclosure is directed to an intravascular nerve modulation system for performing nerve ablation.

Accordingly, one illustrative embodiment includes an intravascular nerve modulation device including an elongate shaft having a proximal end region and a distal end region. An expandable basket having a proximal end region and a distal end region may be affixed to the elongate shaft adjacent to the distal end region of the elongate shaft. The basket may further include an insulating coating disposed over a least a portion of the expandable basket. One or more electrically conductive regions may be disposed along a portion of the distal end region of the expandable basket.

Another illustrative embodiment includes an elongate shaft having a proximal end region and a distal end region. A self-expanding basket including a plurality of interconnected struts forming a network of cells and having a proximal end region and a distal end region, may be affixed to the elongate shaft adjacent to the distal end region of the elongate shaft. The basket may further include an insulating coating disposed over a least a portion of the basket. One or more electrically conductive regions may be disposed along a portion of the distal end region of the basket.

Another illustrative embodiment includes a method for manufacturing a medical device. The method may include coating an expandable basket formed of a conductive material with an insulating material. Selective portions of the insulating material may be removed to define electrically conductive regions.

The above summary of some embodiments is not intended to describe each disclosed embodiment or every implementation of the present disclosure. The Figures, and Detailed Description, which follow, more particularly exemplify these embodiments.

### BRIEF DESCRIPTION OF DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 illustrates a distal portion of an illustrative renal nerve modulation device.

FIG. 3 illustrates an illustrative partial cross-section of the illustrative renal nerve modulation device of FIG. 2.

FIG. 4 illustrates an illustrative expandable basket for use with a nerve modulation system cut open and flattened.

FIG. 5 illustrates another illustrative expandable basket for use with a nerve modulation system cut open and flattened.

FIG. 6 illustrates another illustrative expandable basket for use with a nerve modulation system cut open and flattened.

FIG. 7 illustrates another illustrative expandable basket for use with a nerve modulation system cut open and flattened.

FIG. 8 illustrates another illustrative expandable basket for use with a nerve modulation system cut open and flattened.

FIG. 9 illustrates another illustrative expandable basket for use with a nerve modulation system cut open and flattened.

FIG. 10 illustrates another illustrative expandable basket for use with a nerve modulation system cut open and flattened.

FIG. 11 illustrates another illustrative expandable basket for use with a nerve modulation system cut open and flattened.

FIG. 12 illustrates a portion of another illustrative expandable basket for use with a nerve modulation system.

FIG. 13 illustrates a distal portion of an illustrative renal nerve modulation device.

FIG. 14 illustrates an illustrative expandable basket for use with a nerve modulation system cut open and flattened.

FIG. 15 illustrates another illustrative expandable basket for use with a nerve modulation system cut open and flattened.

FIG. 16 illustrates another illustrative expandable basket for use with a nerve modulation system cut open and flattened.

FIG. 17 illustrates another illustrative expandable basket for use with a nerve modulation system cut open and flattened.

FIG. 18 illustrates another illustrative expandable basket for use with a nerve modulation system cut open and flattened.

FIG. 19 illustrates another illustrative expandable basket for use with a nerve modulation system cut open and flattened.

FIG. 20 illustrates another illustrative expandable basket for use with a nerve modulation system cut open and flattened.

FIG. 21 illustrates another illustrative expandable basket for use with a nerve modulation system cut open and flattened.

FIG. 22 illustrates another illustrative expandable basket for use with a nerve modulation system cut open and flattened.

FIG. 23 illustrates another illustrative expandable basket for use with a nerve modulation system cut open and flattened.

FIG. 24 illustrates another illustrative expandable basket for use with a nerve modulation system cut open and flattened.

FIG. 25 illustrates another illustrative expandable basket for use with a nerve modulation system cut open and flattened.

FIGS. 26A-26D illustrate an illustrative method for forming an expandable basket for use with a nerve modulation system.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

The following description should be read with reference to the drawings, wherein like reference numerals indicate like elements throughout the several views. The drawings, which are not necessarily to scale, are not intended to limit the scope of the claimed invention. The detailed description and drawings illustrate exemplary embodiments of the claimed invention.

All numbers used or otherwise included herein should be considered to be modified by the term “about.” The disclosure or recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular indefinite articles “a,” “an,” and the definite article “the,” should be considered to include or otherwise cover both single and plural referents, unless the content clearly dictates otherwise. In other words, these articles are applicable to one or more referents. As used in this specification and the appended claims, the term “or” should be considered to mean “and/or,” unless the content clearly dictates otherwise.

References in the specification to “an embodiment,” “some embodiments,” “other embodiments,” etc., indicate that the embodiment described may include a particular feature, structure, or characteristic, but every embodiment may not necessarily include the particular feature, structure, or characteristic. Moreover, such phrases do not necessarily refer to the same embodiment. Further, if a particular feature, structure, or characteristic is described in connection with an embodiment, then it would be within the knowledge of one skilled in the art to affect such feature, structure, or characteristic in connection with other embodiments, whether or not explicitly described, unless clearly stated to the contrary.

Certain treatments require the temporary or permanent interruption or modification of select nerve function. One example treatment is renal nerve ablation, which is sometimes used to treat conditions related to hypertension, congestive heart failure, diabetes, or other conditions impacted by high blood pressure or salt retention. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many of the devices and methods are disclosed herein in the context of renal nerve modulation through a blood vessel wall. However, devices and methods of other embodiments may be used in other contexts, such as applications other than where nerve modulation and/or ablation are desired. It is contemplated that the devices and methods may be used in other treatment locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pulmonary vein isolation, tumor ablation, benign prostatic hyperplasia therapy, nerve excitation or blocking or ablation, modulation of muscle activity, hyperthermia or other warming of tissues, etc. The disclosed methods and apparatus can be applied to any relevant medical procedure, involving both human and non-human subjects. The term modulation refers to ablation and other techniques that may alter the function of affected nerves and other tissue. In some embodiments, a single ablation device may be used to sequentially or simultaneously perform multiple ablations, if desired.

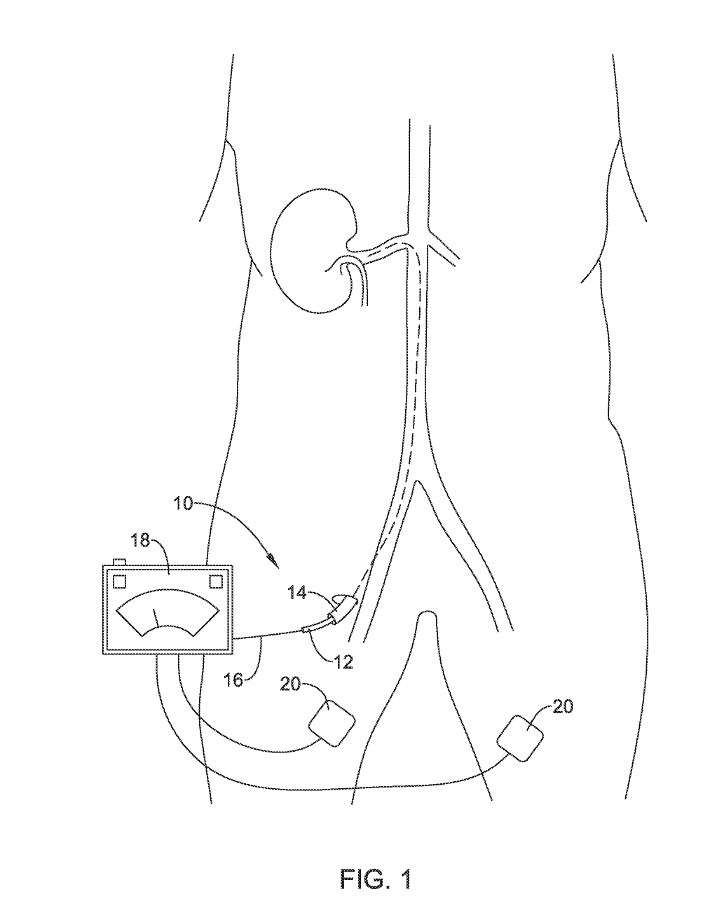

FIG. 1 is a schematic view of an illustrative renal nerve modulation system in situ. The renal nerve modulation system 10 may include one or more conductive element(s) 16 for providing power to a renal nerve modulation device. An illustrative renal nerve modulation device may include an intravascular catheter or nerve modulation device 12 optionally disposed within a delivery sheath or guide catheter 14. The delivery sheath 14 may be adapted to slideably contain the intravascular catheter 12 if a radially expanding distal portion (not shown) of the intravascular catheter 12 is in a non-expanded configuration, as will be discussed in more detail below. A distal end of each of the conductive element(s) 16 is attached to one or more electrodes or electrically conductive regions at a location at or near a distal end of the intravascular catheter 12. A proximal end of each of the conductive element(s) 16 may be connected to a power and control unit 18, which supplies electrical energy used to activate the one or more electrodes. The power and control unit 18 is typically located outside of the patient's body. The electrodes are capable of modulating or ablating tissue upon being suitably activated via the control unit 18. In some instances, return electrode patches 20 may be supplied on the legs or at another conventional location on the patient's body to complete the electrical circuit.

In the following disclosure, the terms energy delivery regions, electrically conductive regions, electrode, and electrodes may be considered to be equivalent to elements capable of ablating adjacent tissue. The disclosure of “adjacent tissue” is intended to cover any tissue located sufficiently proximate the electrode(s) for ablation, and the locations and distances involved are intended to vary depending on application and/or other factors.

The power and control unit 18 may include monitoring elements to monitor parameters, such as power, temperature, voltage, pulse size, impedance and/or shape, and/or other suitable parameters. The power and control unit 18 may also include, or otherwise be used with, sensors mounted along the renal nerve modulation device, as well as suitable controls for performing the desired procedure. In some embodiments, the control unit 18 may control a radio frequency (RF) electrode. The electrode may be configured to operate at a frequency of approximately 460 kHz. However, any desired frequency in the RF range may be used, for example, from 450-500 kHz. In addition, other types of ablation devices may be used as desired including, but not limited to, devices that involve resistance heating, ultrasound, microwave, and laser technologies. The power and control unit 18 may supply different forms of power to these devices.

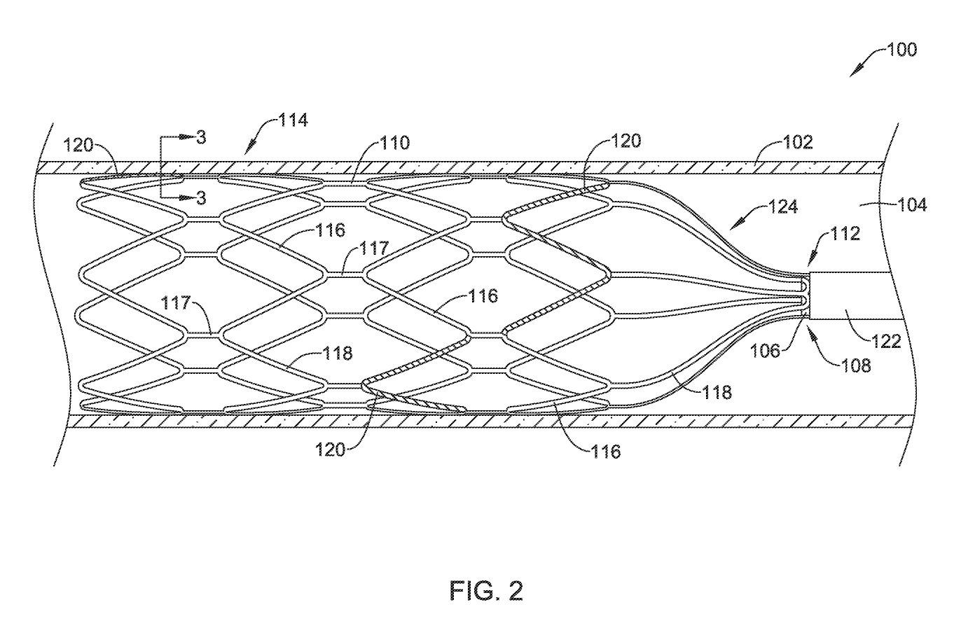

FIG. 2 illustrates a distal portion of an illustrative renal nerve modulating device 100 disposed with a body lumen 104 having a vessel wall 102. Local body tissue (not shown) may surround the vessel wall 102. The local body tissue may comprise adventitia and connective tissues, nerves, fat, fluid, etc., in addition to the muscular vessel wall 102. A portion of the surrounding tissue may constitute the desired treatment region or target tissue. For instance, one or more renal nerves (not shown) may extend along the outer wall of the body lumen 104.

The device 100 may include an elongate catheter shaft 106 having a proximal end (not shown) and a distal end region 108. The elongate shaft 106 may extend proximally from the distal end region 108 to the proximal end configured to remain outside of a patient's body. Although not shown, the proximal end of the elongate shaft 106 may include a hub attached thereto for connecting other treatment devices or providing a port for facilitating other treatments. It is contemplated that the stiffness of the elongate shaft 106 may be modified to form the modulation device 100 for use in various vessel diameters and various locations within the vascular tree.

In some instances, the elongate shaft 106 may have an elongate tubular structure and may include one or more lumens extending therethrough. In some embodiments, the elongate shaft 106 may include one or more guidewire or auxiliary lumens. In some instances, the elongate shaft 106 may include a separate lumen(s) (not shown) for infusion of fluids, such as saline or dye for visualization or for other purposes such as the introduction of a medical device, and so forth. The fluid may facilitate cooling of the modulation device 100 during the ablation procedure, in addition to the cooling of a body lumen. Further, the lumens may be configured in any way known in the art. For example, the lumen(s) may extend along the entire length of the elongate shaft 106 such as in an over-the-wire catheter or may extend only along a distal portion of the elongate shaft 106 such as in a single operator exchange (SOE) catheter. These examples are not intended to be limiting, but rather examples of some possible configurations. While not explicitly shown, the modulation device 100 may further include temperature sensors/wire, an infusion lumen, radiopaque marker bands, fixed guidewire tip, a guidewire lumen, and/or other components to facilitate the use and advancement of the device 100 within the vasculature.

Further, the elongate shaft 106 may have a relatively long, thin, flexible tubular configuration. In some instances, the elongate shaft 106 may have a generally circular cross-section, however, other suitable configurations such as, but not limited to, rectangular, oval, irregular, or the like may also be contemplated. In addition, the elongate shaft 106 may have a cross-sectional configuration adapted to be received in a desired vessel, such as a renal artery. For instance, the elongate shaft 106 may be sized and configured to accommodate passage through an intravascular path, which leads from a percutaneous access site in, for example, the femoral, brachial, or radial artery, to a targeted treatment site, for example, within a renal artery.

The modulation device 100 may further include an expandable basket 110 having a proximal end region 112, a distal end region 114, and an optional bridge segment 124 disposed therebetween. In some instances, in the expanded state, the bridge segment 124 may span between a collapsed proximal end region 112 and the expanded distal end region 114. It is contemplated that the bridge segment 124 have a different pattern or structure than the proximal end region 112 and/or distal end region, as will be discussed in more detail below. In the expanded configuration (shown in FIG. 2), the distal end region 114 may have a larger, generally cylindrical, cross-sectional area than the proximal end region 112.

In some embodiments, the expandable basket 110 may be laser cut from a generally tubular member to form a desired pattern. While the expandable basket 110 is illustrated as having an open cell, generally stent-like, structure it is contemplated that the basket 110 may be formed to have any of a number of different configurations. In some embodiments, the expandable basket 110 may be formed from a plurality of interconnected circumferentially extending struts 116. The struts 116 may have an undulating or serpentine shape, although this is not required. The struts 116 may be connected by one or more connectors 117. It is contemplated that the struts 116 in combination with the connectors 117 may form a cellular configuration with each cell having any shape desired, such as, but not limited to: circular, square, oval, rectangular, polygonal, etc. In some instances, the basket 110 may be formed from a number of generally longitudinally extending tines or may be formed from one or more filaments that may be woven, braided, knotted, etc. These are just examples. It is further contemplated that while the basket 110 is illustrated as including four struts 116 in the distal end region 114, the expandable basket 110 may include any number of struts 116 desired, such as, but not limited to, one, two, three, four, or more. It is further contemplated that the struts 116 may be spaced along the length of the basket 110 from the proximal end region 112 to the distal end region 114 as desired.

It is contemplated that the expandable basket 110 can be made from a number of different materials such as, but not limited to, metals, metal alloys, shape memory alloys and/or polymers, as desired, enabling the basket 110 to be expanded into shape when positioned within the body. For example, the expandable basket 110 can be formed from alloys such as, but not limited to, nitinol or Elgiloy®. Depending on the material selected for construction, the basket 110 may be self-expanding or may require an actuation mechanism, as will be discussed in more detail below. In some embodiments, fibers may be used to make the expandable basket 110, which may be cored fibers, for example, having an outer shell made of nitinol and a platinum core. It is further contemplated that the expandable basket 110 may be formed from or partially from polymers including, but not limited to, polyether ether ketone (PEEK), nylon, polyethylene terephthalate (PET), polyimides, polyether block amides, etc. In some embodiments, the expandable basket 110 may further include radiopaque markers, bands or coatings.

The proximal end region 112 of the basket 110 may be secured to or adjacent to the distal end region 108 of the elongate shaft 106. As noted above, in some instances, the basket 110 may be self-expanding. It is contemplated that a self-expanding basket 110 may be maintained in a compressed (or collapsed) state when an external force is placed on the basket 110. The basket 110 may then expand when the external force is released. In such an instance, the basket 110 may be formed in the expanded state (as shown in FIG. 2) and compressed to fit within a delivery sheath 122. Upon reaching the target location, the delivery sheath 122 can be retracted to deploy the expandable basket 110.

In other embodiments, the device 100 may include an actuation mechanism, for example, a pull wire (not explicitly shown), which may be employed to manipulate or actuate the expandable basket 110 between the collapsed and expanded configurations. In an embodiment, the pull wire may be attached to the proximal end region 112 of the basket 110 such that a push-pull actuation of the pull wire may manipulate the expandable basket 110, thus actuating the expandable basket 110 between the collapsed and expanded configurations.

In some embodiments, the expandable basket 110 may be formed from a conductive material covered with an insulating or semi-insulating coating 118. The expandable basket 110 may be coated with insulating material using any number of coating techniques, such as, but not limited to, dip coating, spray coating, etc. In some instances, the expandable basket 110 may be coated with parylene or other insulating material. In other instances, the coating 118 may be formed of semi-insulating materials, such as but not limited to a porous polymer or a ceramic. It is further contemplated that the coating 118 may be a very thin polymer or coating.

It is contemplated that the coating 118 may be removed from, or not applied to, one or more locations on the expandable basket 110 to form one or more electrically conductive regions 120 configured to deliver RF energy to the target region around the vessel wall 102. The one or more electrically conductive regions 120 may function as one or more electrodes for delivering RF energy to a desired treatment area, although this is not required. In some instances, the one or more electrically conductive regions 120 may be discrete elements or electrodes affixed to the basket 110.

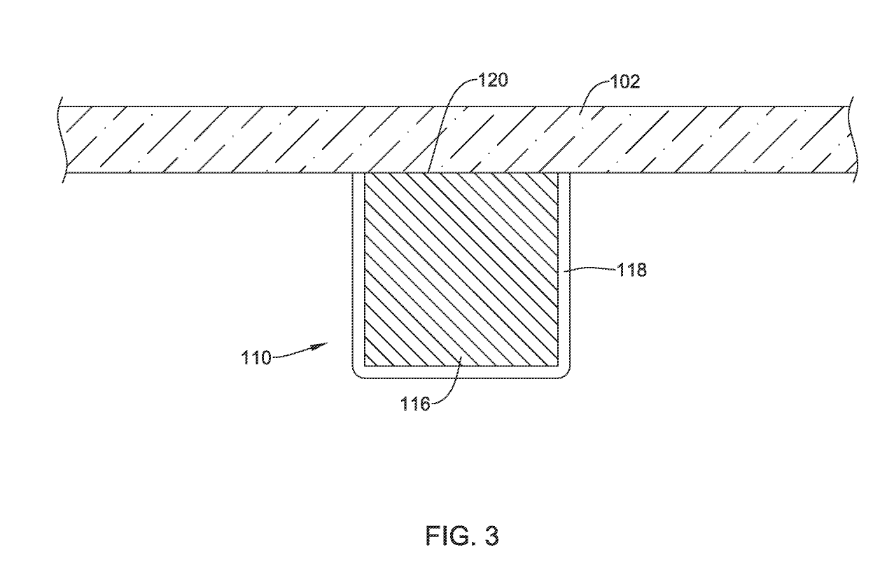

In the expanded configuration, one or more electrically conductive regions 120 may contact the vessel wall 102. For example, FIG. 3 illustrates a cross-section of an illustrative conductive region 120 contacting a vessel wall 102 taken at line 3-3 in FIG. 2. As can be seen, the coating 118 is absent from an outer surface of the expandable basket 110 to form an electrically conductive region 120 positioned to contact a vessel wall 102. While FIG. 3 illustrates the coating 118 as absent from only the side of the basket 110 contacting the vessel wall 102 (the outer surface of the basket 110), it is contemplated that the coating 118 can be removed from as many sides as desired to provide the desired ablation energy. It is further contemplated that the coating 118 may be absent on any single side (or combination of sides) desired. For example, in some instances, the coating 118 may be present on a portion of the basket 110 contacting the vessel wall 102 but absent on any one of the sides not in contact with the wall 102 such that the energy delivery region 120 does not contact the vessel wall 102 directly. In some instances, the coating 118 may be absent from an inner surface of the expandable basket 110. While the strut 116 is illustrated as having a generally square cross-sectional shape, it is contemplated that the cross-section of the struts 116 may be any shape desired, such as but not limited to, square, circular, oval, etc.

Referring again to FIG. 2, it is contemplated that the modulation device 100 may be advanced through the vasculature to a desired treatment region, such as the renal artery. The modulation device 100 may be advanced with the expandable basket 110 in a collapsed position. For example, the delivery sheath 122 may be disposed over the basket 110 to maintain the basket 110 in a collapsed position. When the expandable basket 110 is positioned adjacent to the target treatment region, the delivery sheath 122 may be retracted to allow at least a portion of the expandable basket 110 to contact the vessel wall 102. As discussed above, pull wires, or other actuation mechanisms can be used in place of or in combination with the delivery sheath 122 to facilitate delivery of the device 100. In some embodiments, a guide catheter or vascular access catheter may be used in combination with the delivery sheath 122 to facilitate advancement of the device 100. When the expandable basket 110 is in the expanded configuration, the proximal end region 112 may remain in a generally collapsed or low-profile configuration. In the expanded configuration, the outer surface of the expandable basket 110, including electrically conductive regions 120, may come into gentle contact with the vessel wall 102.

One or more electrical conductors (not explicitly shown) may connect the expandable basket 110 to a power and control unit which provides RF energy to the expandable basket 110. In some instances, RF energy may be supplied to the entire basket 110, but is only emitted from the electrically conductive regions 120. In other instances, RF energy may be supplied to the electrically conductive regions 120 via conductive traces (not explicitly shown) disposed in or on the basket 110. The conductive traces may connect the electrically conductive regions 120 to a power and control unit. It is contemplated that the electrically conductive regions 120 may function as multiple electrodes connected in parallel to deliver RF energy to the desired treatment region. For example, a single-channel control unit may provide power to the electrically conductive regions 120 simultaneously. This may allow for multi-point ablation while reducing procedure time compared to performing sequential ablation of discrete spots. It is further contemplated that simultaneous ablation of multiple treatment locations may also avoid or reduce overlapping treatment areas or widely separated treatment areas. In some instances, overlapping treatment areas may cause locally severe damage to the vessel or other adjacent tissue. Widely separated treatment areas may leave untreated nerves, making the therapy less effective. In some instances, providing the electrically conductive regions 120 on an inner surface of the expandable basket 110 may also reduce damage to peripheral tissues while providing consistent positioning of the conductive regions 120.

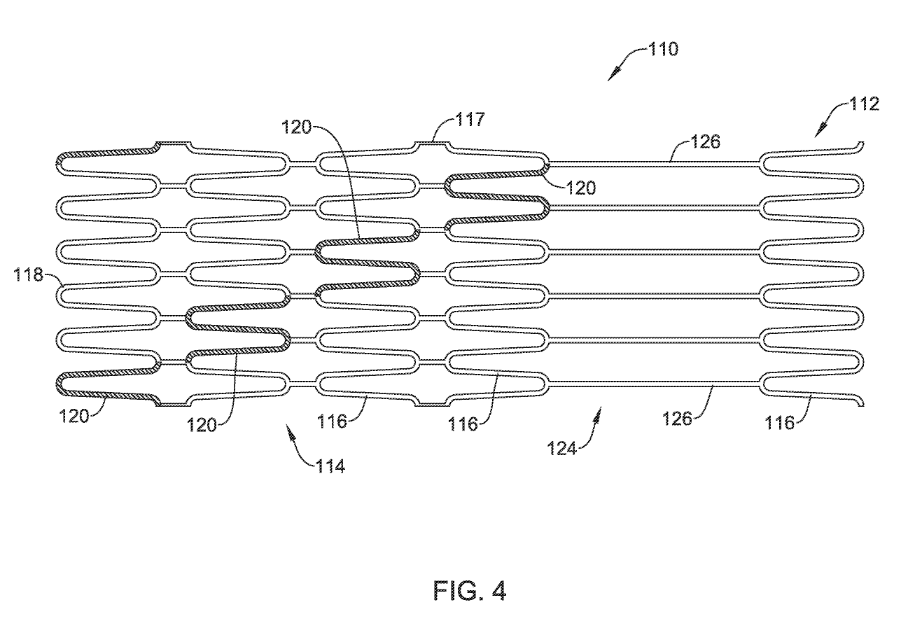

It is contemplated that the electrically conductive regions 120 may be formed about the expandable basket 110 in any manner desired to provide the desired ablation pattern. FIG. 4 illustrates the expandable basket 110 of FIG. 2 cut open and flattened for clarity of illustration. As shown in FIG. 4, the electrically conductive regions 120 may be formed such that each conductive region 120 is longitudinally and circumferentially spaced from the adjacent region(s) 120 thus forming a generally helical pattern when the expandable basket 110 is in the expanded configuration. While the expandable basket 110 is illustrated as having four electrically conductive regions 120, it is contemplated that the basket may include any number of conductive regions 120 desired, such as, but not limited to, one, two, three, four, or more.

As shown in FIG. 4, the proximal end region 112 and the distal end region 114 may include a plurality of interconnected struts 116 forming a generally cell-like pattern in combination with connectors 117. In some instances, the proximal end region 112 and the distal end region 114 may be interconnected by an optional bridge segment 124 including any number of generally longitudinally extending struts 126. It is contemplated that in some instances, the bridge segment 124 may be omitted. In such an instance, the electrically conductive regions 120 may be positioned such that they are on a portion of the distal end region 114 of the basket 110 that is fully expanded when the basket 110 is in the expanded configuration. It is further contemplated that the electrically conductive regions 120 may be on the outside, inside, side surfaces, or any combination thereof, of the struts 116 or connectors 117 as desired.

It is contemplated that various basket 110 geometries may be utilized. For example, the basket 110 can include more or fewer interconnecting struts 116, connectors 117, etc. In some instances, the basket geometry can be chosen to obtain greater or lesser flexibility. Baskets with more rows or rings can be used to spread out the heated areas along a longer length, at the expense of requiring a longer landing zone in the artery and having a longer stiff section, especially during introduction and positioning. It is further contemplated that various patterns of insulated and non-insulated areas can be utilized to achieve the desired geometry of ablation. For example, the depth, volume, and temperature required for the treatment of a target tissue zone may affect the electrode (electrically conductive region) configuration that is required. The size and positioning of electrically conductive regions 120 can be chosen to produce a more evenly heated zone, for example, such as by using a greater electrode area of non-wall-contact electrode(s) and a lesser area of wall contact electrode(s). Alternatively, an uneven or asymmetric heating zone can be obtained if desired.

The electrically conductive regions 120 can be positioned so that they are reliably positioned as desired in the lumen 104 when the distal end region 114 of the basket 110 expands. The configuration of the electrically conductive regions or electrodes 120 may be chosen to obtain the desired heating of a volume of tissue. Wall-contact electrodes 120 may provide more concentrated heating of nearby tissues, and non-wall-contact electrodes 120 provide less concentrated heating of deeper tissues. Electrically conductive regions 120 can be arranged in single or multiple contiguous areas, contiguous strips, circumferential rings, helical lines, or discrete spots, positioned closer or farther from each other and with various surface areas along the inside and/or outside of the basket 110 to shape the heated zone as needed. Repeated patterns or combinations of arrangements can be used. Greater heating can be provided towards one end, or toward the middle, or along one side, compared to other areas, for example.

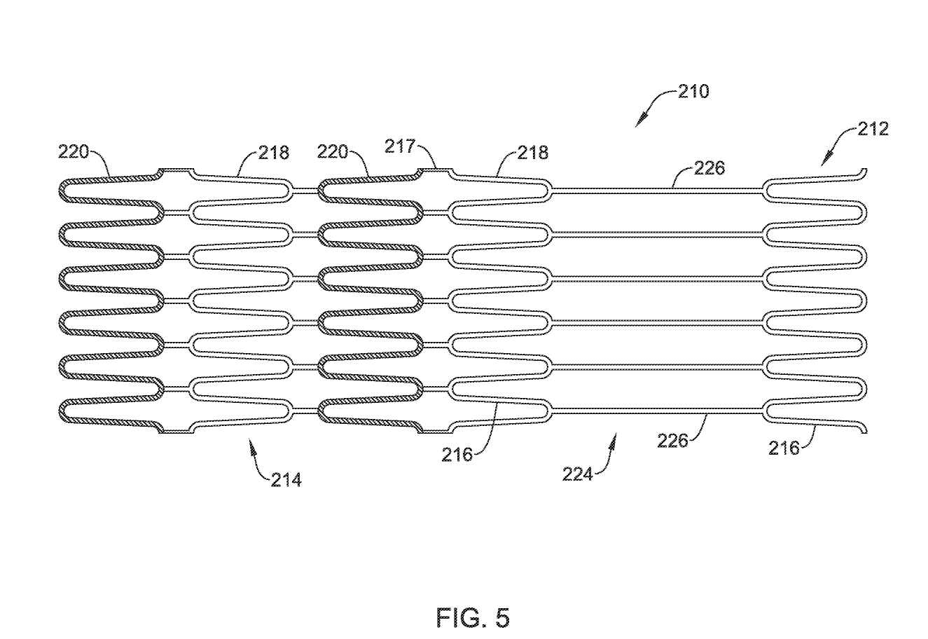

FIG. 5 illustrates another illustrative expandable basket 210 cut open and flattened for clarity of illustration. It is contemplated that the expandable basket 210 may be used in combination with any of the modulation systems disclosed herein. As shown in FIG. 5, electrically conductive regions 220 may be formed such that each conductive region 220 extends around a circumference of the basket 210. In some instances, conductive regions 220 may be alternately formed with non-conductive or coated regions 218 disposed therebetween. The electrically conductive regions 220 may form a generally ring-like configuration extending about a circumference of the basket 210 when the expandable basket 210 is in the expanded configuration. While the expandable basket 210 is illustrated as having two electrically conductive regions 220, it is contemplated that the basket may include any number of conductive regions 220 desired, such as, but not limited to, one, two, three, four, or more. It is further contemplated that the electrically conductive regions 220 may be on the outside, inside, or side surfaces, or any combination thereof, of the struts 216 or connectors 217 as desired.

As shown in FIG. 5, the proximal end region 212 and the distal end region 214 may include a plurality of interconnected struts 216 forming a generally cell-like pattern in combination with connectors 217. In some instances, the proximal end region 212 and the distal end region 214 may be interconnected by an optional bridge segment 224 including any number of generally longitudinally extending struts 226. It is contemplated that in some instances, the bridge segment 224 may be omitted. In such an instance, the electrically conductive regions 220 may be positioned such that they are on a portion of the distal end region 214 of the basket 210 that is fully expanded when the basket 210 is in the expanded configuration.

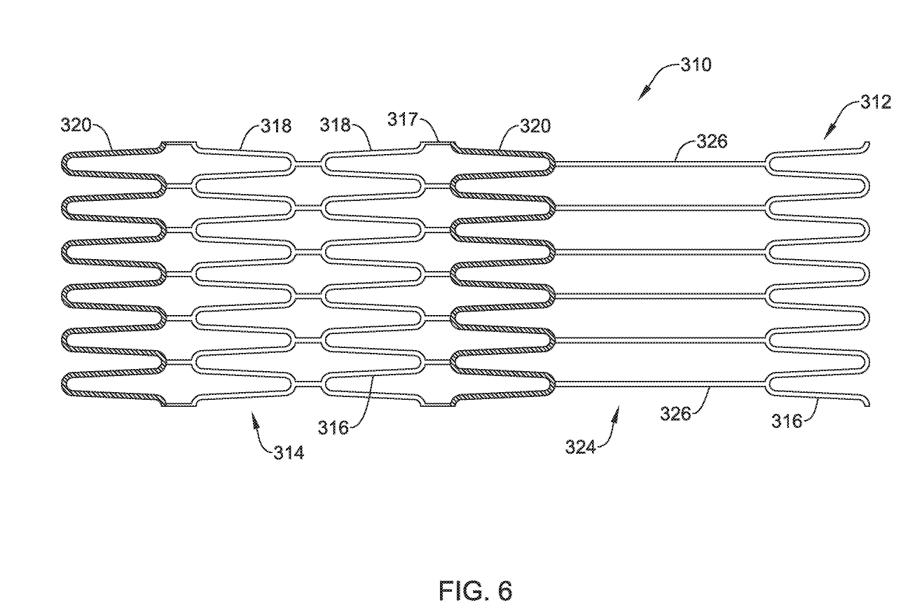

FIG. 6 illustrates another illustrative expandable basket 310 cut open and flattened for clarity of illustration. It is contemplated that the expandable basket 310 may be used in combination with any of the modulation systems disclosed herein. As shown in FIG. 6, electrically conductive regions 320 may be formed such that each conductive region 320 extends around a circumference of the basket 310. In some instances, conductive regions 320 may be longitudinally spaced from one another by two or more non-conductive or coated regions 318. The electrically conductive regions 320 may form a generally ring-like configuration when the expandable basket 310 is in the expanded configuration. While the expandable basket 310 is illustrated as having two electrically conductive regions 320, it is contemplated that the basket may include any number of conductive regions 320 desired, such as, but not limited to, one, two, three, four, or more. It is further contemplated that the electrically conductive regions 320 may be on the outside, inside, or side surfaces, or any combination thereof, of the struts 316 or connectors 317 as desired.

As shown in FIG. 6, the proximal end region 312 and the distal end region 314 may include a plurality of interconnected struts 316 forming a generally cell-like pattern in combination with connectors 317. In some instances, the proximal end region 312 and the distal end region 314 may be interconnected by an optional bridge segment 324 including any number of generally longitudinally extending struts 326. It is contemplated that in some instances, the bridge segment 324 may be omitted. In such an instance, the electrically conductive regions 320 may be positioned such that they are on a portion of the distal end region 314 of the basket 310 that is fully expanded when the basket 310 is in the expanded configuration.

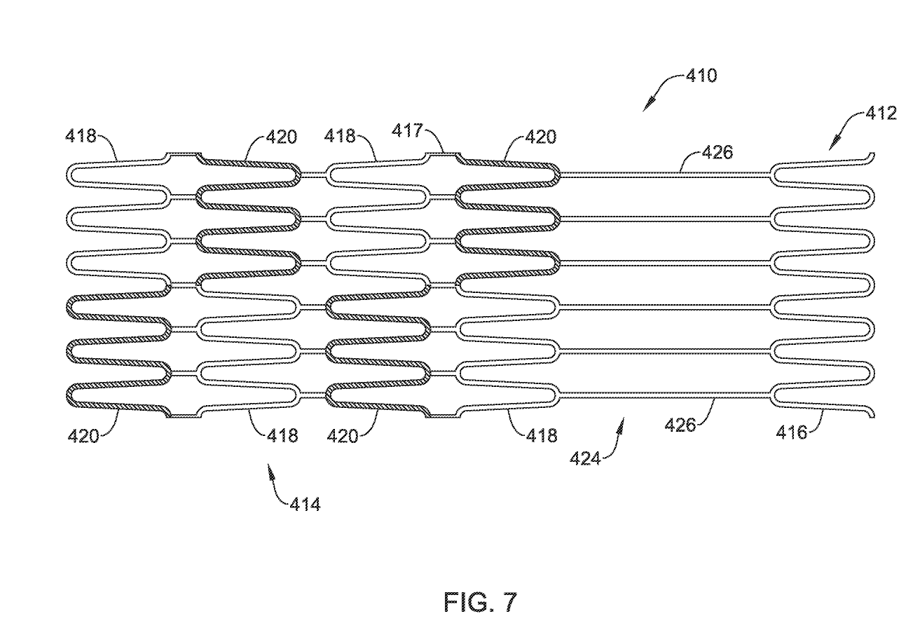

FIG. 7 illustrates another illustrative expandable basket 410 cut open and flattened for clarity of illustration. It is contemplated that the expandable basket 410 may be used in combination with any of the modulation systems disclosed herein. As shown in FIG. 7, electrically conductive regions 420 may be formed such that each conductive region 420 is longitudinally and circumferentially spaced from the adjacent region(s) 420 by non-conductive or coated regions 418 thus forming a generally helical pattern when the expandable basket 410 is in the expanded configuration. In some instances, the electrically conductive regions 420 may extend around approximately half of the circumference of the basket 410, although this is not required. While the expandable basket 410 is illustrated as having four electrically conductive regions 420, it is contemplated that the basket may include any number of conductive regions 420 desired, such as, but not limited to, one, two, three, four, or more. It is further contemplated that the electrically conductive regions 420 may be on the outside, inside, or side surfaces, or any combination thereof, of the struts 416 or connectors 417 as desired.

As shown in FIG. 7, the proximal end region 412 and the distal end region 414 may include a plurality of interconnected struts 416 forming a generally cell-like pattern in combination with connectors 417. In some instances, the proximal end region 412 and the distal end region 414 may be interconnected by an optional bridge segment 424 including any number of generally longitudinally extending struts 426. It is contemplated that in some instances, the bridge segment 424 may be omitted. In such an instance, the electrically conductive regions 420 may be positioned such that they are on a portion of the distal end region 414 of the basket 410 that is fully expanded when the basket 410 is in the expanded configuration.

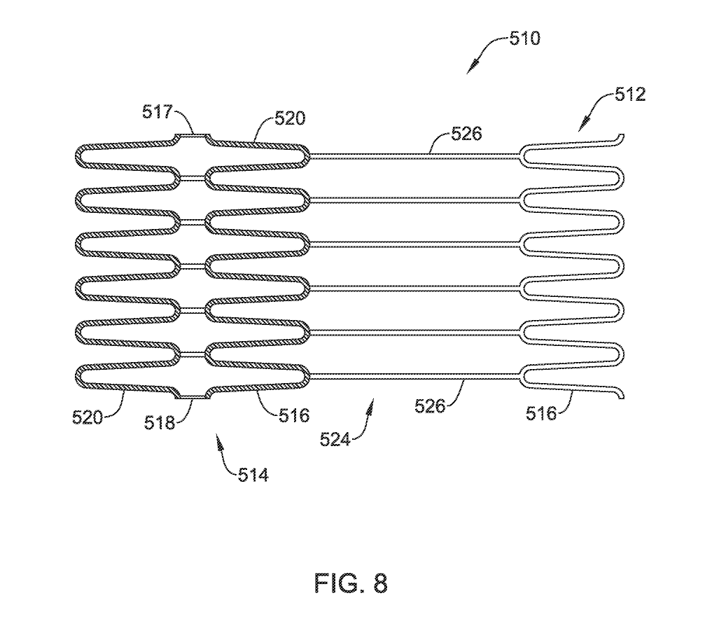

FIG. 8 illustrates another illustrative expandable basket 510 cut open and flattened for clarity of illustration. It is contemplated that the expandable basket 510 may be used in combination with any of the modulation systems disclosed herein. As shown in FIG. 8, electrically conductive regions 520 may be formed such that each conductive region 520 extends around a circumference of the basket 510. In some instances, conductive regions 520 may be longitudinally spaced from one another by connectors 517 including an insulating or semi-insulating coating 518. While the expandable basket 510 is illustrated as having two electrically conductive regions 520, it is contemplated that the basket may include any number of conductive regions 520 desired, such as, but not limited to, one, two, three, four, or more. It is further contemplated that the electrically conductive regions 520 may be on the outside, inside, or side surfaces, or any combination thereof, of the struts 516 or connectors 517 as desired.

As shown in FIG. 8, the proximal end region 512 and the distal end region 514 may include a plurality of interconnected struts 516 forming a generally cell-like pattern in combination with connectors 517. In some instances, the proximal end region 512 and the distal end region 514 may be interconnected by an optional bridge segment 524 including any number of generally longitudinally extending struts 526. It is contemplated that in some instances, the bridge segment 524 may be omitted. In such an instance, the electrically conductive regions 520 may be positioned such that they are on a portion of the distal end region 514 of the basket 510 that is fully expanded when the basket 510 is in the expanded configuration.

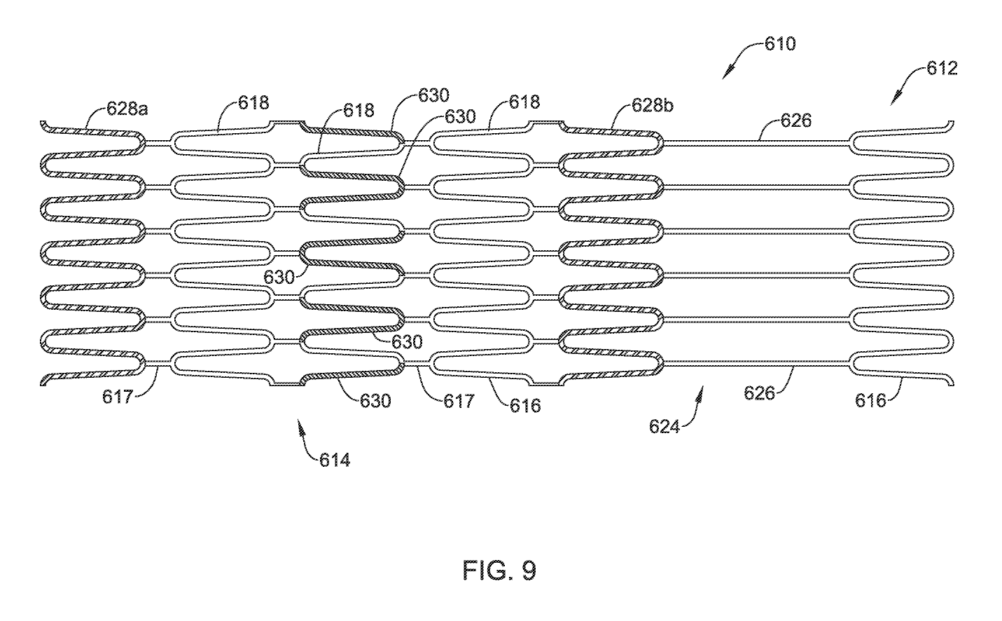

FIG. 9 illustrates another illustrative expandable basket 610 cut open and flattened for clarity of illustration. It is contemplated that the expandable basket 610 may be used in combination with any of the modulation systems disclosed herein. In some instances the expandable basket 610 may include one or more electrically conductive regions 628a, 628b (collectively 628) on the inside surface of the basket 610 and one or more electrically conductive regions 630 on the outside surface of the basket 610. This may provide the benefits of both wall contact and non-wall-contact ablation. In some instances, the one or more electrically conductive regions 628 on the inside surface of the basket 610 may extend around a circumference of the basket 610. However, this is not required. It is contemplated that the one or more electrically conductive regions 628 on the inside surface of the basket 610 may be circumferentially and/or longitudinally spaced about the basket 610. In some embodiments, the one or more electrically conductive regions 630 on the outside surface of the basket 610 may be longitudinally positioned between the one or more electrically conductive regions 628a, 628b on the inside surface of the basket 610, although this is not required. In some instances, the one or more electrically conductive regions 630 on the outside surface of the basket 610 may be circumferentially and/or longitudinally spaced about the basket 610 as desired. The conductive regions 628, 630 may be separated by one or more regions including an insulating or semi-insulating coating 618. It is contemplated that the basket 610 may include any number of conductive regions 628, 630 desired, such as, but not limited to, one, two, three, four, or more. It is further contemplated that the electrically conductive regions 628, 630 may be on the outside, inside, or side surfaces, or any combination thereof, of the struts 616 or connectors 617 as desired.

As shown in FIG. 9, the proximal end region 612 and the distal end region 614 may include a plurality of interconnected struts 616 forming a generally cell-like pattern in combination with connectors 617. In some instances, the proximal end region 612 and the distal end region 614 may be interconnected by an optional bridge segment 624 including any number of generally longitudinally extending struts 626. It is contemplated that in some instances, the bridge segment 624 may be omitted. In such an instance, the electrically conductive regions 628, 630 may be positioned such that they are on a portion of the distal end region 614 of the basket 610 that is fully expanded when the basket 610 is in the expanded configuration.

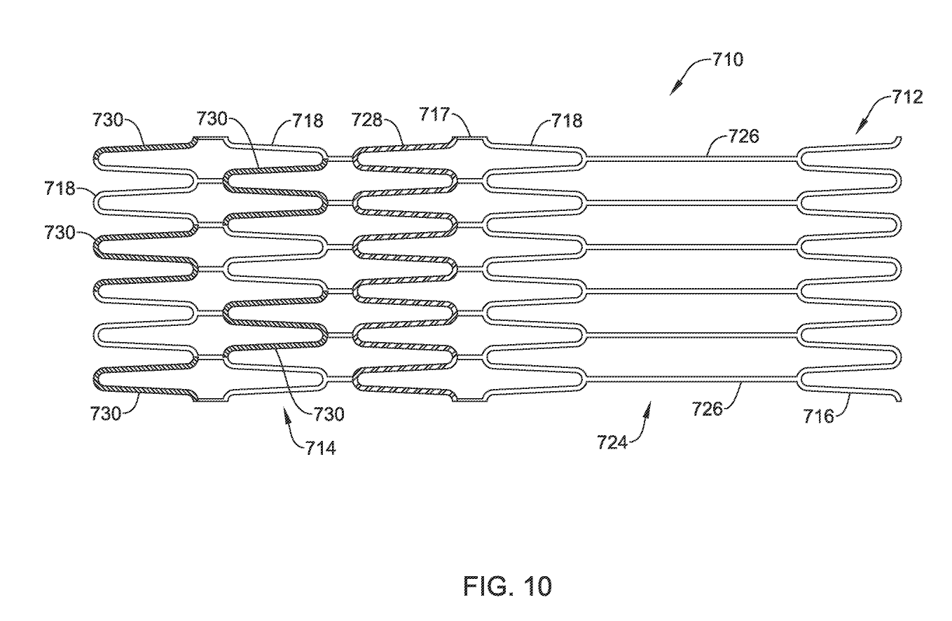

FIG. 10 illustrates another illustrative expandable basket 710 cut open and flattened for clarity of illustration. It is contemplated that the expandable basket 710 may be used in combination with any of the modulation systems disclosed herein. In some instances the expandable basket 710 may include one or more electrically conductive regions 728 on the inside surface of the basket 710 and one or more electrically conductive regions 730 on the outside surface of the basket 710. This may provide the benefits of both wall contact and non-wall-contact ablation. In some instances, the one or more electrically conductive regions 728 on the inside surface of the basket 710 may extend around a circumference of the basket 710. However, this is not required. It is contemplated that the one or more electrically conductive regions 728 on the inside surface of the basket 710 may be circumferentially and/or longitudinally spaced about the basket 710 and separated by one or more regions including an insulating or semi-insulating coating 718. In some embodiments, the one or more electrically conductive regions 730 on the outside surface of the basket 710 may be distal to the one or more electrically conductive regions 728 on the inside surface of the basket 710, although this is not required. In some instances, the one or more electrically conductive regions 730 on the outside surface of the basket 710 may be circumferentially and/or longitudinally spaced about the basket 710 and separated by one or more regions including an insulating or semi-insulating coating 718 as desired. It is contemplated that the basket 710 may include any number of conductive regions 728, 730 desired, such as, but not limited to, one, two, three, four, or more. It is further contemplated that the electrically conductive regions 728, 730 may be on the outside, inside, or side surfaces, or any combination thereof, of the struts 716 or connectors 717 as desired.

As shown in FIG. 10, the proximal end region 712 and the distal end region 714 may include a plurality of interconnected struts 716 forming a generally cell-like pattern in combination with connectors 717. In some instances, the proximal end region 712 and the distal end region 714 may be interconnected by an optional bridge segment 724 including any number of generally longitudinally extending struts 726. It is contemplated that in some instances, the bridge segment 724 may be omitted. In such an instance, the electrically conductive regions 728, 730 may be positioned such that they are on a portion of the distal end region 714 of the basket 710 that is fully expanded when the basket 710 is in the expanded configuration.

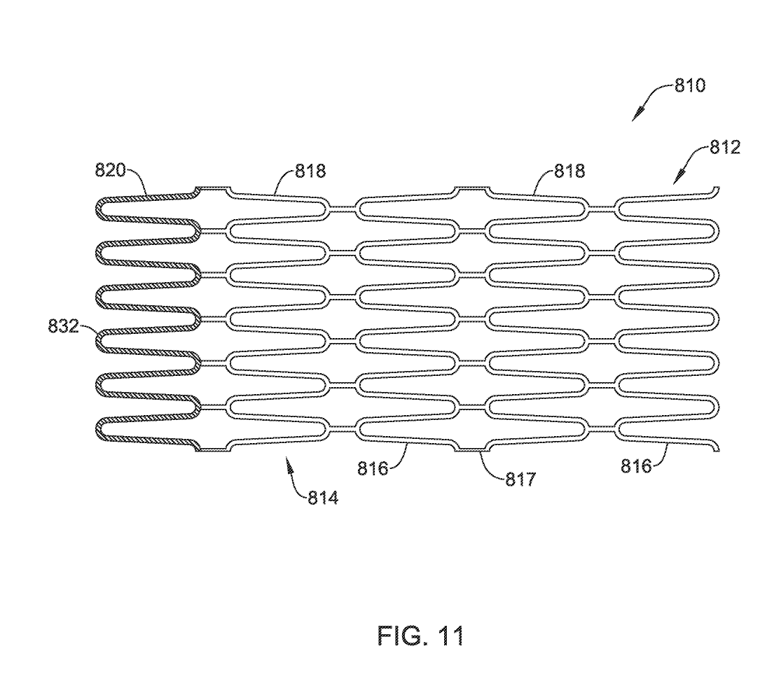

FIG. 11 illustrates another illustrative expandable basket 810 cut open and flattened for clarity of illustration. It is contemplated that the expandable basket 810 may be used in combination with any of the modulation systems disclosed herein. In some embodiments, the expandable basket 810 may be formed from a material covered with an insulating or semi-insulating coating 818. Portions of the coating 818 may be removed or omitted to form electrically conductive regions 820. As shown in FIG. 11, electrically conductive regions 820 may be formed such that the conductive region 820 extends around a circumference of the basket 810. In some instances, the conductive regions 820 may be disposed adjacent a distal end 832 of the basket 810. While the expandable basket 810 is illustrated as having one electrically conductive region 820, it is contemplated that the basket may include any number of conductive regions 820 desired, such as, but not limited to, one, two, three, four, or more. It is further contemplated that the electrically conductive regions 820 may be on the outside, inside, or side surfaces, or any combination thereof, of the struts 816 or connectors 817 as desired.

As shown in FIG. 11, the proximal end region 812 and the distal end region 814 may include a plurality of interconnected struts 816 forming a generally cell-like pattern in combination with connectors 817. In some instances, the electrically conductive regions 820 may be positioned such that they are on a portion of the distal end region 814 of the basket 810 that is fully expanded when the basket 810 is in the expanded configuration. It is further contemplated that in the absence of a bridge segment, the basket 810 may include additional struts 816 to lengthen the basket 810 and allow the distal end region 814 and/or the electrically conductive regions 820 to contact the vessel wall.

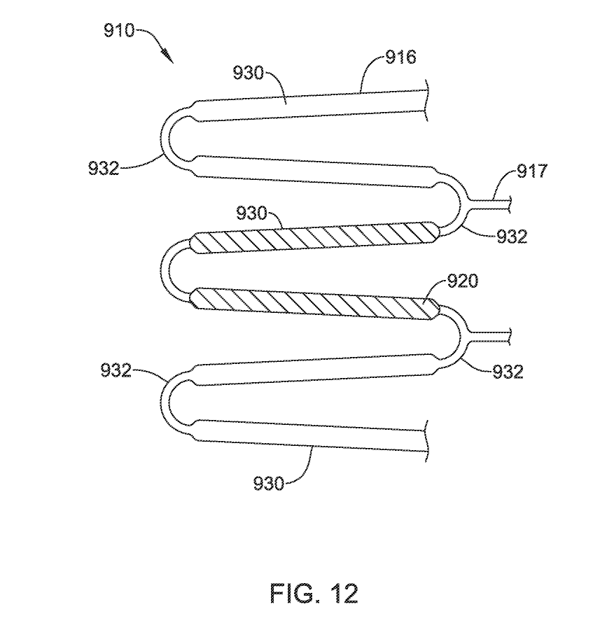

FIG. 12 illustrates a portion of another illustrative expandable basket 910 including one or more electrically conductive regions 920. It is contemplated that the expandable basket 910 may be used in combination with any of the modulation systems or baskets disclosed herein. It is further contemplated that while only a portion of basket 910 is illustrated, it may be similar in form and function to any of the baskets described herein. While not explicitly shown, the basket 910 may include a proximal end region and a distal end region. In some embodiments, the expandable basket 910 may be formed from one or more interconnected circumferentially extending struts 916. The struts 916 may be connected by one or more connectors 917. It is contemplated that the struts 916 in combination with the connectors 917 may form a cellular configuration. The electrically conductive regions 920 can be positioned about the basket 910 so that they are reliably positioned as desired in the vessel when the distal end region of the basket 910 expands. In some embodiments, the electrically conductive regions 920 may be on the outside, inside, side surfaces, or any combination thereof, of the struts 916 or connectors 917 as desired. It is contemplated that the electrically conductive regions 920 may be formed about the expandable basket 910 in any manner desired to provide the desired ablation pattern.

In some embodiments, the struts 916 may have a generally serpentine shape including relatively straight portions 930 and curved portions 932 joining adjacent straight portions 930. The curved portions, or hinges, 932 may have a smaller cross-sectional area than the straight portions 930. The radial expansion force of the basket may relate generally to strut width. In some configurations, the hinge 932 structure can beneficially reduce the radial expansion force since the basket 910 only needs to hold the electrically conductive regions 920 in apposition with the vessel wall and not provide a stenting expansion force to the vessel. The smaller hinges 932 may thus result in a lower expansion force of the basket 910. It is further contemplated that a wider or larger straight portion 930 may provide sufficient surface area to the electrically conductive regions 920 to reduce thermal tissue damage and/or fouling of the electrically conductive region.

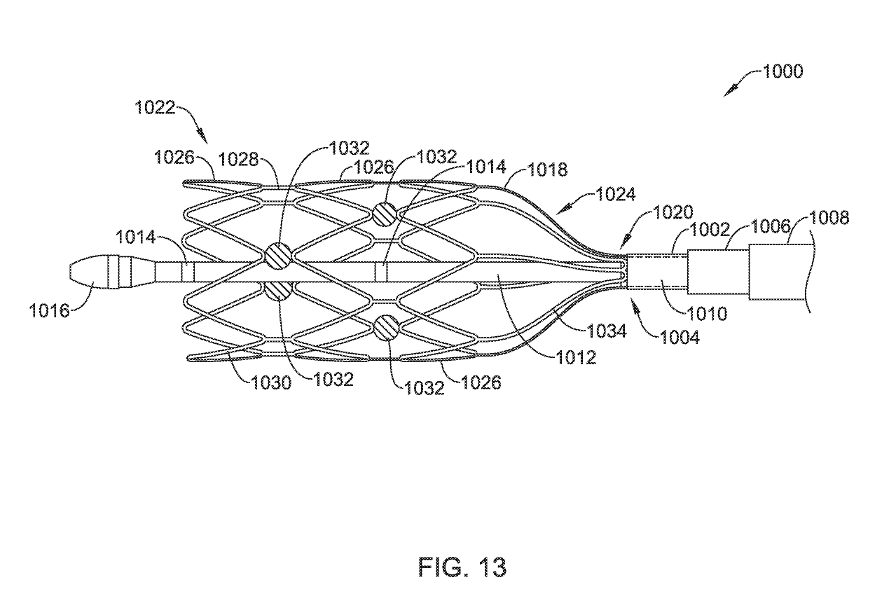

FIG. 13 illustrates a distal portion of another illustrative renal nerve modulating system 1000. The modulation system 1000 may be similar in form and function to modulation system 100. The system 1000 may include an elongate catheter shaft 1002 having a proximal end (not shown) and a distal end region 1004. The elongate shaft 1002 may extend proximally from the distal end region 1004 to the proximal end configured to remain outside of a patient's body. Although not shown, the proximal end of the elongate shaft 1002 may include a hub attached thereto for connecting other treatment devices or providing a port for facilitating other treatments. It is contemplated that the stiffness of the elongate shaft 1002 may be modified to form a modulation system 1000 for use in various vessel diameters and various locations within the vascular tree.

In some instances, the elongate shaft 1002 may have an elongate tubular structure and may include one or more lumens 1010 extending therethrough. In some embodiments, the elongate shaft 1002 may include one or more guidewire or auxiliary lumens. In some instances, the elongate shaft 1002 may include a separate lumen(s) (not shown) for infusion of fluids, such as saline or dye for visualization or for other purposes such as the introduction of a medical device, and so forth. The fluid may facilitate cooling of the modulation system 1000 during the ablation procedure, in addition to the cooling of a body lumen. Further, the lumens may be configured in any way known in the art. For example, the lumen(s) may extend along the entire length of the elongate shaft 1002 such as in an over-the-wire catheter or may extend only along a distal portion of the elongate shaft 1002 such as in a single operator exchange (SOE) catheter. These examples are not intended to be limiting, but rather examples of some possible configurations. While not explicitly shown, the modulation system 1000 may further include temperature sensors/wire, an infusion lumen, radiopaque marker bands, fixed guidewire tip, a guidewire lumen, and/or other components to facilitate the use and advancement of the system 1000 within the vasculature.

Further, the elongate shaft 1002 may have a relatively long, thin, flexible tubular configuration. In some instances, the elongate shaft 1002 may have a generally circular cross-section, however, other suitable configurations such as, but not limited to, rectangular, oval, irregular, or the like may also be contemplated. In addition, the elongate shaft 1002 may have a cross-sectional configuration adapted to be received in a desired vessel, such as a renal artery. For instance, elongate shaft 1002 may be sized and configured to accommodate passage through an intravascular path, which leads from a percutaneous access site in, for example, the femoral, brachial, or radial artery, to a targeted treatment site, for example, within a renal artery.

The modulation system 1000 may include a delivery sheath 1006 and/or a guide catheter 1008 for facilitating advancement of the system 1000 through the vasculature. In some embodiments, the system 1000 may further include a support tube and/or guidewire 1012. The support/guidewire tube 1012 may include one or more radiopaque marker bands 1014 to facilitate placement of the system 1000. The support/guidewire tube 1012 may further include an atraumatic tip 1016.

The modulation system 1000 may further include an expandable basket 1018 having a proximal end region 1020, a distal end region 1022, and an optional bridge segment 1024 disposed therebetween. In some instances, in the expanded state, the bridge segment 1024 may span between a collapsed proximal end region 1020 and the expanded distal end region 1022. It is contemplated that the bridge segment 1024 may have a different pattern or structure than the proximal end region 1020 or distal end region 1022. For example, the bridge segment may include any number of generally longitudinally extending struts 1034 while the proximal end region 1020 and the distal end region 1022 may include any number of circumferentially extending struts. This is just an example. In the expanded configuration (shown in FIG. 13), the distal end region 1022 may have a larger, generally cylindrical, cross-sectional area than the collapsed proximal end region 1020.

In some embodiments, the expandable basket 1018 may be laser cut from a generally tubular member to form a desired pattern. While the expandable basket 1018 is illustrated as having an open cell, generally stent-like, structure it is contemplated that the basket 1018 may be formed to have any of a number of different configurations. In some embodiments, the expandable basket 1018 may be formed from a plurality of interconnected circumferentially extending struts 1026. The struts 1026 may be connected by one or more connectors 1028. It is contemplated that the struts 1026 in combination with the connectors 1028 may form a cellular configuration with each cell having any shape desired, such as, but not limited to: circular, square, oval, rectangular, polygonal, etc. In some instances, the basket 1018 may be formed from a number of generally longitudinally extending tines or may be formed from one or more filaments that may be woven, braided, knotted, etc. These are just examples. It is further contemplated that while basket 1018 is illustrated as including three struts 1026 in the distal end region 1022, the expandable basket 1018 may include any number of struts 1026 desired, such as, but not limited to, one, two, three, four, or more. It is further contemplated that the struts 1026 may be spaced from the proximal end region 1020 to the distal end region 1022 as desired.

It is contemplated that the expandable basket 1018 can be made from a number of different materials such as, but not limited to, metals, metal alloys, shape memory alloys and/or polymers, as desired, enabling the basket 1018 to be expanded into shape when positioned within the body. For example, the expandable basket 1018 can be formed from alloys such as, but not limited to, nitinol or Elgiloy®. Depending on the material selected for construction, the basket 1018 may be self-expanding or may require an actuation mechanism as will be discussed in more detail below. In some embodiments, fibers may be used to make the expandable basket 1018, which may be cored fibers, for example, having an outer shell made of nitinol and a platinum core. It is further contemplated that the expandable basket 1018 may be formed from or partially from polymers including, but not limited to, polyether ether ketone (PEEK), nylon, polyethylene terephthalate (PET), polyimides, polyether block amides, etc. In some embodiments, the expandable basket 1018 may further include radiopaque markers, bands or coatings.

The proximal end region 1020 of the basket 1018 may be secured to or adjacent to the distal end region 1004 of the elongate shaft 1002. As noted above, in some instances, the basket 1018 may be self-expanding. It is contemplated that a self-expanding basket 1018 may be maintained in a compressed (or collapsed state) when an external force is placed on the basket 1018. The basket 1018 may then expand when the external force is released. In such an instance, the basket 1018 may be formed in the expanded state (as shown in FIG. 13) and compressed to fit within a delivery sheath 1006. Upon reaching the target location, the delivery sheath 1006 can be retracted to deploy the expandable basket 1018.

In other embodiments, the system 1000 may include an actuation mechanism, for example, a pull wire (not explicitly shown), which may be employed to manipulate or actuate the expandable basket 1018 between the collapsed and expanded configurations. In an embodiment, the pull wire may be attached to the proximal end region 1020 of the basket 1018 such that a push-pull actuation of the pull wire may manipulate the expandable basket 1018, thus actuating the expandable basket 1018 between the collapsed and expanded configurations.

In some embodiments, the expandable basket 1018 may be formed from a conductive material covered with an insulating or semi-insulating coating 1030. The expandable basket 1018 may be coated with insulating material using any number of coating techniques, such as, but not limited to, dip coating, spray coating, etc. In some instances, the expandable basket 1018 may be coated with parylene or other insulating material. In some instances, the coating 1130 may be formed of semi-insulating materials, such as, but not limited to a porous polymer or a ceramic. It is further contemplated that the coating 1030 may be a very thin polymer or coating.

It is contemplated that the coating 1030 may be removed from or not applied to one or more locations on the expandable basket 1018 to form one or more electrode pads or electrically conductive regions 1032 configured to deliver RF energy to a target region. In some instances, the one or more electrically conductive regions 1032 may be discrete elements or electrodes affixed to the basket 1018. The one or more electrically conductive regions 1032 may function as one or more electrodes for delivering RF energy to a desired treatment area. In the expanded configuration, one or more electrically conductive regions 1032 may contact a vessel wall. For example, the coating 1030 may be absent from an outer surface of the expandable basket 1018 to form an electrically conductive region 1032 positioned to contact a vessel wall. However, it is contemplated that the coating 1030 can be removed from as many sides of the basket 1018 (inner, outer, side surfaces, etc.) as desired to provide the desired ablation energy. It is further contemplated that the coating 1030 may be absent on any single side (or combination of sides) desired. For example, in some instances, the coating 1030 may be present on a portion of the basket 1018 contacting the vessel wall but absent on any one of the sides not in contact with the wall such that the energy delivery region 1032 does not contact the vessel wall directly. In some instances, the insulating material 1030 may be absent from an inner surface of the expandable basket 1018. It is contemplated that strut 1026 may have a generally square cross-sectional shape, however the cross-section of the struts 1026 may be any shape desired. In some instances, such as, but not limited to, when a non-conductive basket 1018 is utilized, the electrically conductive regions 1032 may be separately formed of an electrically conductive material and attached to the basket 1018.

In some embodiments, the electrically conductive regions 1032 may be enlarged pad regions relative to other portions of the basket 1018. In some instances, a small electrode surface area may result in higher temperatures at the electrode which may cause increased vessel wall injury, blood damage, and/or fouling of the electrode surface. It is contemplated that the electrically conductive regions 1032 may be round, oblong, square, rectangular, polygonal, or other shape as desired. In some embodiments, the enlarged electrically conductive regions 1032 may be positioned between the circumferential struts 1026. In other embodiments, the electrically conductive regions 1032 may be formed from or on a portion of the circumferential struts 1026. It is contemplated that the size, position, and/or spacing of the electrically conductive regions 1032 may affect the power required to ablate the target tissue as well as the geometry of the heated zone. For example, fewer electrodes, greater spacing, and/or smaller electrodes may require greater power. It is contemplated that the electrically conductive regions 1032 may be formed or positioned about the expandable basket 1018 in any manner desired to provide the desired ablation pattern. As shown in FIG. 13, the electrically conductive regions 1032 may be spaced about the circumference and the length of the basket 1018. In some instances, the electrically conductive regions 1032 may form circumferential rings, although this is not required.

The modulation system 1000 may be advanced through the vasculature to a desired treatment region, such as the renal artery. The modulation system 1000 may be advanced with the expandable basket 1018 in a collapsed position. For example, the delivery sheath 1006 may be disposed over the basket 1018 to maintain the basket 1018 in a collapsed position. When the expandable basket 1018 is positioned adjacent to the target treatment region, the delivery sheath 1006 may be retracted to allow at least a portion of the expandable basket 1018 to contact the vessel wall. As discussed above, pull wires, or other actuation mechanisms can be used in place of or in combination with the delivery sheath 1006 to facilitate delivery of the system 1000. It is contemplated that the expandable basket 1018 may be formed such that it is sufficient to obtain contact with the vessel wall, but not strong enough to significantly expand the vessel. In some embodiments, a guide catheter 1008 or vascular access catheter may be used in combination with the delivery sheath 1006 to facilitate advancement of the system 1000. When the expandable basket 1018 is in the expanded configuration, the proximal end region 1020 may remain in a generally collapsed or low-profile configuration. In the expanded configuration, the outer surface of the expandable basket 1018, sometimes including electrically conductive regions 1032, may come into gentle contact with the vessel wall.

One or more electrical conductors (not explicitly shown) may connect the expandable basket 1018 to a power and control unit which provides RF energy to the expandable basket 1018. In some instances, RF energy may be supplied to the entire basket 1018, but is only emitted from the electrically conductive regions 1032. In other instances, RF energy may be supplied to the electrically conductive regions 1032 via conductive traces (not explicitly shown) disposed in or on the basket 1018. The conductive traces may connect the electrically conductive regions 1032 to a power and control unit. It is contemplated that the electrically conductive regions 1032 may function as multiple electrodes connected in parallel to deliver RF energy to the desired treatment region. For example, a single-channel control unit may provide power to the electrically conductive regions 1032 simultaneously. This may allow for multi-point ablation while reducing procedure time compared to performing sequential ablation of discrete spots. It is further contemplated that simultaneous ablation of multiple treatment locations may also avoid or reduce overlapping treatment areas or widely separated treatment areas. In some instances, overlapping treatment areas may cause locally severe damage to the vessel or other adjacent tissue. Widely separated treatment areas may leave untreated nerves, making the therapy less effective. In some instances, providing the electrically conductive regions 1032 on an inner surface of the expandable basket 1018 may also reduce damage to peripheral tissues while providing consistent positioning of the conductive regions 1032, but may also cause increased blood heating.

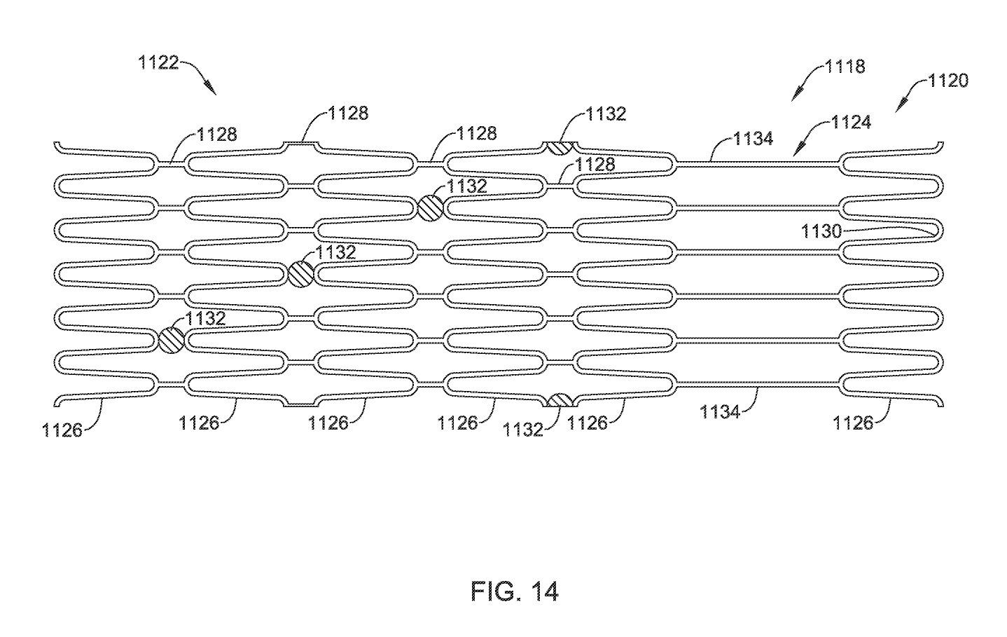

FIG. 14 illustrates another illustrative expandable basket 1118 cut open and flattened for clarity of illustration. In some embodiments, the expandable basket 1118 may be formed from a conductive material covered with an insulating or semi-insulating coating 1130. Portions of the coating 1130 may be removed or omitted to form enlarged electrode pads or electrically conductive regions 1132. It is contemplated that the electrically conductive regions 1132 may be round, oblong, square, rectangular, polygonal, or other shape as desired. In some embodiments, the enlarged electrically conductive regions 1132 may be positioned between the circumferential struts 1126. In other embodiments, the electrically conductive regions 1132 may be formed from or on a portion of the circumferential struts 1126. As shown in FIG. 14, the electrically conductive regions 1132 may be formed such that each conductive region 1132 is longitudinally and circumferentially spaced from the adjacent region(s) 1132 thus forming a generally helical pattern when the expandable basket 1118 is in the expanded configuration. While the expandable basket 1118 is illustrated as having four electrically conductive regions 1132, it is contemplated that the basket may include any number of conductive regions 1132 desired, such as, but not limited to, one, two, three, four, or more.

As shown in FIG. 14, the proximal end region 1120 and the distal end region 1122 may include a plurality of interconnected struts 1126 forming a generally cell-like pattern in combination with connectors 1128. In some instances, the proximal end region 1120 and the distal end region 1122 may be interconnected by an optional bridge segment 1124 including any number of generally longitudinally extending struts 1134. It is contemplated that in some instances, the bridge segment 1124 may be omitted. In such an instance, the electrically conductive regions 1132 may be positioned such that they are on a portion of the distal end region 1122 of the basket 1118 that is fully expanded when the basket 1118 is in the expanded configuration. It is further contemplated that the electrically conductive regions 1132 may be on the outside, inside, or side surfaces, or any combination thereof, of the struts 1126 or connectors 1128 as desired.

It is contemplated that various basket 1118 geometries may be utilized. For example, the basket 1118 can include more or fewer interconnecting struts 1126, connectors 1128, etc. In some instances, the basket geometry can be chosen to obtain greater or lesser flexibility. Baskets with more rows or rings of struts 1126 can be used to spread out the heated areas more, at the expense of requiring a longer landing zone in the artery, and having a longer stiff section, especially during introduction and positioning. It is further contemplated that various patterns of insulated and non-insulated areas can be utilized to achieve the desired geometry of ablation. For example, the depth, volume, and temperature required for the treatment of a target tissue zone may affect the electrode (electrically conductive region) configuration that is required. The size and positioning of electrically conductive regions 1132 can be chosen to produce a more even heated zone, for example, such as by using a greater electrode area of non-wall-contact electrode(s) and a lesser area of wall contact electrode(s). Alternatively, an uneven or asymmetric heating zone can be obtained if desired.

The electrically conductive regions 1132 can be arranged so that they are reliably positioned as desired in the vessel when the distal end region 1122 of the basket 1118 expands. The configuration of the electrically conductive regions or electrodes 1132 may be chosen to obtain the desired heating of a volume of tissue. Wall-contact electrodes 1132 may provide more concentrated heating of nearby tissues, and non-wall-contact electrodes 1132 may provide less concentrated heating of deeper tissues. Electrically conductive regions 1132 can be arranged in in single or multiple contiguous areas, contiguous strips, circumferential rings, helical lines, or discrete spots, positioned closer or farther from each other and with various surface areas along the inside and/or outside of the basket 1118 to shape the heated zone as needed. Repeated patterns or combinations of arrangements can be used. Greater heating can be provided towards one end, or toward the middle, or along one side, compared to other areas, for example.

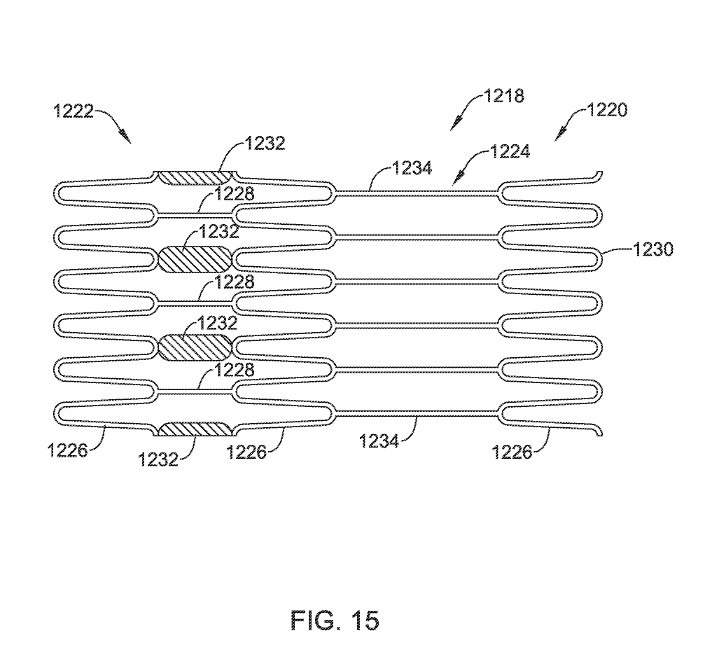

FIG. 15 illustrates another illustrative expandable basket 1218 cut open and flattened for clarity of illustration. It is contemplated that the expandable basket 1218 may be used in combination with any of the modulation systems disclosed herein. In some embodiments, the expandable basket 1218 may be formed from a conductive material covered with an insulating or semi-insulating coating 1230. Portions of the coating 1230 may be removed or omitted to form enlarged electrode pads or electrically conductive regions 1232. As shown in FIG. 15, electrically conductive regions 1232 may be formed as generally oblong regions between adjacent struts 1226. In some instances, the electrically conductive regions may be alternately formed with insulated connectors 1228 disposed therebetween. The electrically conductive regions 1232 may be disposed about a circumference of the basket 1218 to generally provide ablative energy around the circumference of a vessel. While the expandable basket 1218 is illustrated as having three electrically conductive regions 1232, it is contemplated that the basket may include any number of conductive regions 1232 desired, such as, but not limited to, one, two, three, four, or more. It is further contemplated that the electrically conductive regions 1232 may be on the outside, inside, or side surfaces, or any combination thereof, of the struts 1226 or connectors 1228 as desired.

As shown in FIG. 15, the proximal end region 1220 and the distal end region 1222 may include a plurality of interconnected struts 1226 forming a generally cell-like pattern in combination with connectors 1228. It is contemplated that any number of struts 1226 and/or connectors 1228 may be used to form the basket 1218. In some instances, the proximal end region 1220 and the distal end region 1222 may be interconnected by an optional bridge segment 1224 including any number of generally longitudinally extending struts 1234. It is contemplated that in some instances, the bridge segment 1224 may be omitted. In such an instance, the electrically conductive regions 1232 may be positioned such that they are on a portion of the distal end region 1222 of the basket 1218 that is fully expanded when the basket 1218 is in the expanded configuration.

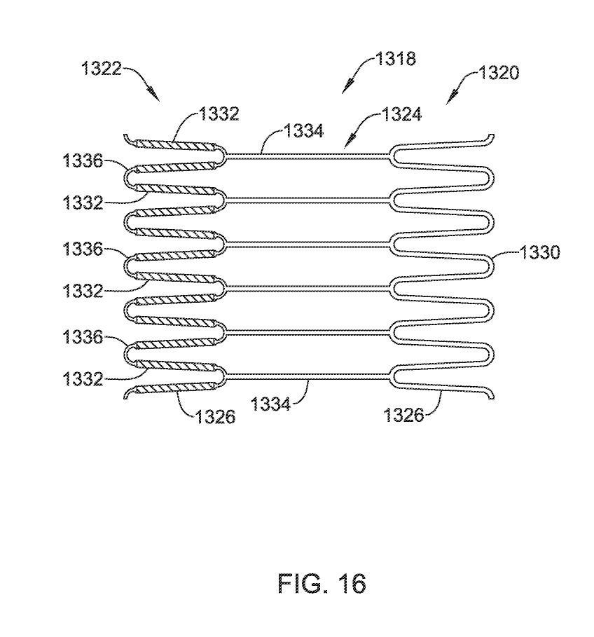

FIG. 16 illustrates another illustrative expandable basket 1318 cut open and flattened for clarity of illustration. It is contemplated that the expandable basket 1318 may be used in combination with any of the modulation systems disclosed herein. In some embodiments, the expandable basket 1318 may be formed from a conductive material covered with an insulating or semi-insulating coating 1330. Portions of the coating 1330 may be removed or omitted to form enlarged electrode pads or electrically conductive regions 1332. As shown in FIG. 16, electrically conductive regions 1332 may be formed as generally enlarged regions of the struts 1326. The electrically conductive regions 1332 may be interconnected by a plurality of curved elements 1336 forming a generally serpentine shape. The curved elements 1336 may have a smaller cross-section or diameter than the electrically conductive regions 1332. The electrically conductive regions 1332 may be disposed about a circumference of the basket 1318 to generally provide ablative energy around the circumference of a vessel. While the expandable basket 1318 is illustrated as having twelve electrically conductive regions 1332, it is contemplated that the basket may include any number of conductive regions 1332 desired, such as, but not limited to, one, two, three, four, or more. It is further contemplated that the electrically conductive regions 1332 may be on the outside, inside, or side surfaces, or any combination thereof, of the struts 1326 or connectors (when so provided) as desired.

As shown in FIG. 16, the proximal end region 1320 and the distal end region 1322 may include a one or more interconnected struts 1326 forming a generally cell-like pattern. It is contemplated that any number of struts 1326 and/or connectors may be used to form the basket 1318. In some instances, the proximal end region 1320 and the distal end region 1322 may be interconnected by an optional bridge segment 1324 including any number of generally longitudinally extending struts 1334. It is contemplated that in some instances, the bridge segment 1324 may be omitted. In such an instance, the electrically conductive regions 1332 may be positioned such that they are on a portion of the distal end region 1322 of the basket 1318 that is fully expanded when the basket 1318 is in the expanded configuration.

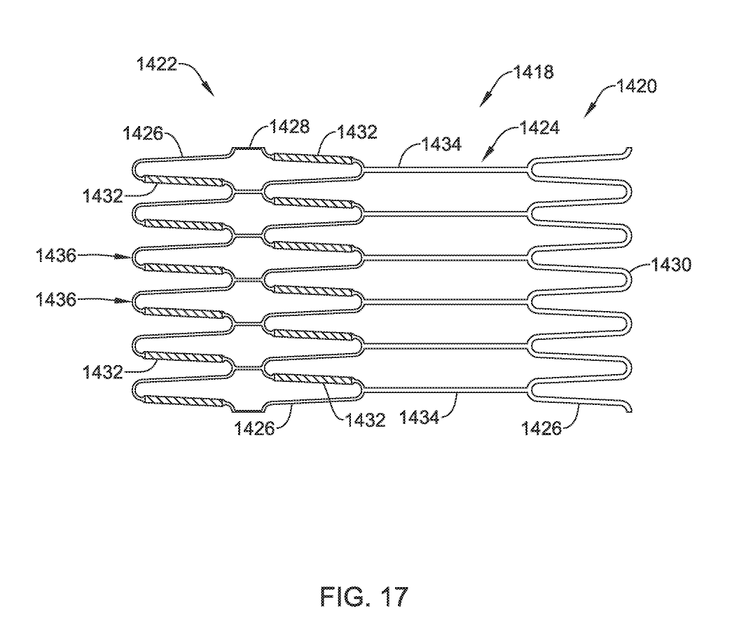

FIG. 17 illustrates another illustrative expandable basket 1418 cut open and flattened for clarity of illustration. It is contemplated that the expandable basket 1418 may be used in combination with any of the modulation systems disclosed herein. In some embodiments, the expandable basket 1418 may be formed from a conductive material covered with an insulating or semi-insulating coating 1430. Portions of the coating 1430 may be removed or omitted to form enlarged electrode pads or electrically conductive regions 1432. As shown in FIG. 17, electrically conductive regions 1432 may be formed as generally enlarged regions of the struts 1426. The electrically conductive regions 1432 may be interconnected by a plurality of curved elements 1436 forming a generally serpentine shape. The curved elements 1436 may have a smaller cross-section or diameter than the electrically conductive regions 1432. In some instances, other portions of the struts 1426 may also or alternatively have a smaller cross-section or diameter than the electrically conductive regions 1432. The electrically conductive regions 1432 may be separated by insulated regions of the struts 1426 or regions of the struts 1426 including coating 1430. The electrically conductive regions 1432 may be disposed about a circumference of the basket 1418 to generally provide ablative energy around the circumference of a vessel. It is contemplated that the electrically conductive regions 1432 on a strut 1426 may be circumferentially staggered with the electrically conductive regions 1432 on an adjacent strut 1426. While the expandable basket 1418 is illustrated as having twelve electrically conductive regions 1432, it is contemplated that the basket may include any number of conductive regions 1432 desired, such as, but not limited to, one, two, three, four, or more. It is further contemplated that the electrically conductive regions 1432 may be on the outside, inside, or side surfaces, or any combination thereof, of the struts 1426 or connectors 1428 as desired.

As shown in FIG. 17, the proximal end region 1420 and the distal end region 1422 may include a one or more interconnected struts 1426 forming a generally cell-like pattern in combination with connectors 1428. It is contemplated that any number of struts 1426 and/or connectors 1428 may be used to form the basket 1418. In some instances, the proximal end region 1420 and the distal end region 1422 may be interconnected by an optional bridge segment 1424 including any number of generally longitudinally extending struts 1434. It is contemplated that in some instances, the bridge segment 1424 may be omitted. In such an instance, the electrically conductive regions 1432 may be positioned such that they are on a portion of the distal end region 1422 of the basket 1418 that is fully expanded when the basket 1418 is in the expanded configuration.

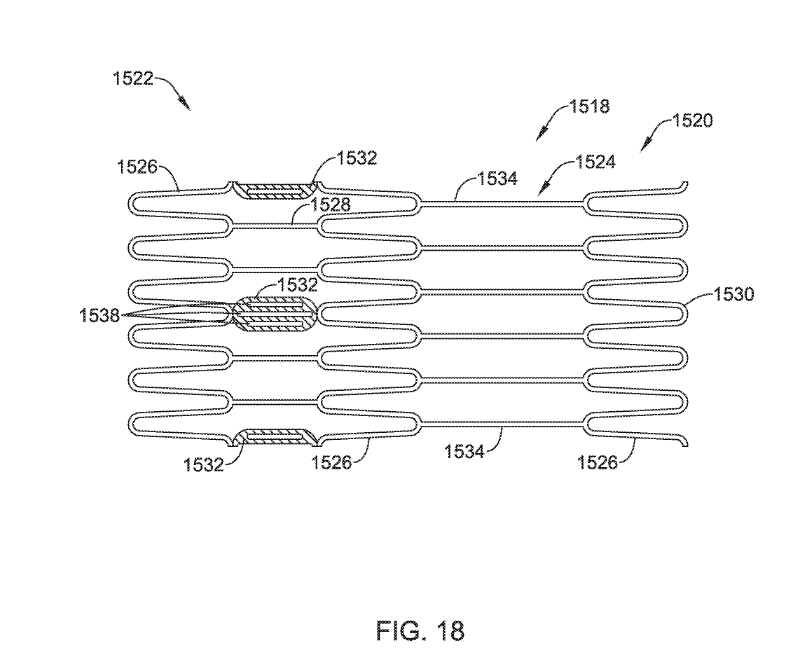

FIG. 18 illustrates another illustrative expandable basket 1518 cut open and flattened for clarity of illustration. It is contemplated that the expandable basket 1518 may be used in combination with any of the modulation systems disclosed herein. In some embodiments, the expandable basket 1518 may be formed from a conductive material covered with an insulating or semi-insulating coating 1530. Portions of the coating 1530 may be removed or omitted to form enlarged electrode pads or electrically conductive regions 1532. As shown in FIG. 18, the electrically conductive regions 1532 may be formed as generally oblong regions between adjacent struts 1526. In some instances, the electrically conductive regions 1532 may be alternately formed with insulated connectors 1528 disposed therebetween. The electrically conductive regions 1532 may be disposed about a circumference of the basket 1518 to generally provide ablative energy around the circumference of a vessel. While the expandable basket 1518 is illustrated as having two electrically conductive regions 1532, it is contemplated that the basket may include any number of conductive regions 1532 desired, such as, but not limited to, one, two, three, four, or more.

It is contemplated that the outer surface of the basket 1518 (e.g. the surface capable of contacting a vessel wall) may be entirely coated with insulation 1530 while the electrically conductive regions 1532 are formed on an inner surface of the basket 1518. This may position the electrically conductive regions 1532 a short distance from the vessel wall when the basket 1518 is deployed. However, this is not required. It is contemplated that the electrically conductive regions may be positioned on any surface of the basket 1518 desired. In some embodiments, the electrically conductive regions 1532 may include one or more fenestrations or openings 1538. It is contemplated that openings 1538 take any shape desired such as, but not limited, longitudinal slots, holes, or apertures. In some instances, openings 1538 in the enlarged electrically conductive regions 1532 may provide additional access for electrical current to flow from the electrically conductive regions 1532 to the target tissue. This may provide more consistent off-wall heating by providing more uniform current path lengths from the electrically conductive regions 1532 to the vessel wall. It is contemplated that in the absence of openings 1538, current flowing from large electrically conductive regions or electrodes in an off-wall configuration may be less consistent due to the longer path from the center of the electrode than from the edges, which may lead to uneven heating. In some embodiments, the openings 1538 may also facilitate cooling of the vessel wall tissue at the electrically conductive regions 1532 by allowing heat transfer to the flowing blood.

As shown in FIG. 18, the proximal end region 1520 and the distal end region 1522 may include a plurality of interconnected struts 1526 forming a generally cell-like pattern in combination with connectors 1528. In some instances, the proximal end region 1520 and the distal end region 1522 may be interconnected by an optional bridge segment 1524 including any number of generally longitudinally extending struts 1534. It is contemplated that in some instances, the bridge segment 1524 may be omitted. In such an instance, the electrically conductive regions 1532 may be positioned such that they are on a portion of the distal end region 1522 of the basket 1518 that is fully expanded when the basket 1518 is in the expanded configuration. It is further contemplated that the electrically conductive regions 1532 may be on the outside, inside, or side surfaces, or any combination thereof, of the struts 1526 or connectors 1528 as desired.

It is contemplated that various basket 1518 geometries may be utilized. For example, the basket 1518 can include more or fewer interconnecting struts 1526, connectors 1528, etc. In some instances, the basket geometry can be chosen to obtain greater or lesser flexibility. Baskets with more rows or rings of struts 1526 can be used to spread out the heated areas more, at the expense of requiring a longer landing zone in the artery, and having a longer stiff section, especially during introduction and positioning. It is further contemplated that various patterns of insulated and non-insulated areas can be utilized to achieve the desired geometry of ablation. For example, the depth, volume, and temperature required for the treatment of a target tissue zone may affect the electrode (electrically conductive region) configuration that is required. The size and positioning of electrically conductive regions 1532 can be chosen to produce a more even heated zone, for example, such as by using a greater electrode area of non-wall-contact electrode(s) and a lesser area of wall contact electrode(s). Alternatively, an uneven or asymmetric heating zone can be obtained if desired.

The electrically conductive regions 1532 can be arranged so that they are reliably positioned as desired in the vessel when the distal end region 1522 of the basket 1518 expands. The configuration of the electrically conductive regions or electrodes 1532 may be chosen to obtain the desired heating of a volume of tissue. Wall-contact electrodes 1532 may provide more concentrated heating of nearby tissues and non-wall-contact electrodes 1532 may provide less concentrated heating of deeper tissues. Electrically conductive regions 1532 can be arranged in single or multiple contiguous areas, contiguous strips, circumferential rings, helical lines, or discrete spots, positioned closer or farther from each other and with various surface areas along the inside and/or outside of the basket 1518 to shape the heated zone as needed. Repeated patterns or combinations of arrangements can be used. Greater heating can be provided towards one end, or toward the middle, or along one side, compared to other areas, for example. Openings 1538 in the electrically conductive regions 1532 may also be arranged as desired to provide the desired treatment geometry.

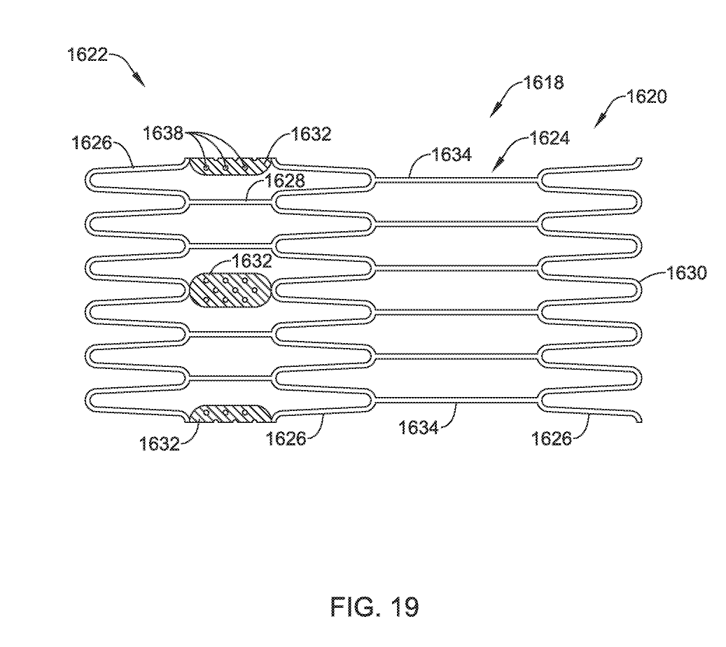

FIG. 19 illustrates another illustrative expandable basket 1618 cut open and flattened for clarity of illustration. It is contemplated that the expandable basket 1618 may be used in combination with any of the modulation systems disclosed herein. In some embodiments, the expandable basket 1618 may be formed from a conductive material covered with an insulating or semi-insulating coating 1630. Portions of the coating 1630 may be removed or omitted to form enlarged electrode pads or electrically conductive regions 1632. As shown in FIG. 19, electrically conductive regions 1632 may be formed as the electrically conductive regions 1632 may be formed as generally oblong regions between adjacent struts 1626. In some instances, the electrically conductive regions 1632 may be alternately formed with insulated connectors 1628 disposed therebetween. The electrically conductive regions 1632 may be disposed about a circumference of the basket 1618 to generally provide ablative energy around the circumference of a vessel. While the expandable basket 1618 is illustrated as having two electrically conductive regions 1632, it is contemplated that the basket may include any number of conductive regions 1632 desired, such as, but not limited to, one, two, three, four, or more.

It is contemplated that the outer surface of the basket 1618 (e.g. the surface capable of contacting a vessel wall) may be entirely coated with insulation 1630 while the electrically conductive regions 1632 are formed on an inner surface of the basket 1618. This may position the electrically conductive regions 1632 a short distance from the vessel wall when the basket 1618 is deployed. However, this is not required. It is contemplated that the electrically conductive regions may be positioned on any surface of the basket 1618 desired. In some embodiments, the electrically conductive regions 1632 may include one or more fenestrations or openings 1638. It is contemplated that openings 1638 take any shape desired such as, but not limited, longitudinal slots, holes, or apertures. In some instances, openings 1638 in the enlarged electrically conductive regions 1632 may provide additional access for electrical current to flow from the electrically conductive regions 1632 to the target tissue.

As shown in FIG. 19, the proximal end region 1620 and the distal end region 1622 may include a one or more interconnected struts 1626 forming a generally cell-like pattern in combination with connectors 1628. It is contemplated that any number of struts 1626 and/or connectors 1628 may be used to form the basket 1618. In some instances, the proximal end region 1620 and the distal end region 1622 may be interconnected by an optional bridge segment 1624 including any number of generally longitudinally extending struts 1634. It is contemplated that in some instances, the bridge segment 1624 may be omitted. In such an instance, the electrically conductive regions 1632 may be positioned such that they are on a portion of the distal end region 1622 of the basket 1618 that is fully expanded when the basket 1618 is in the expanded configuration.

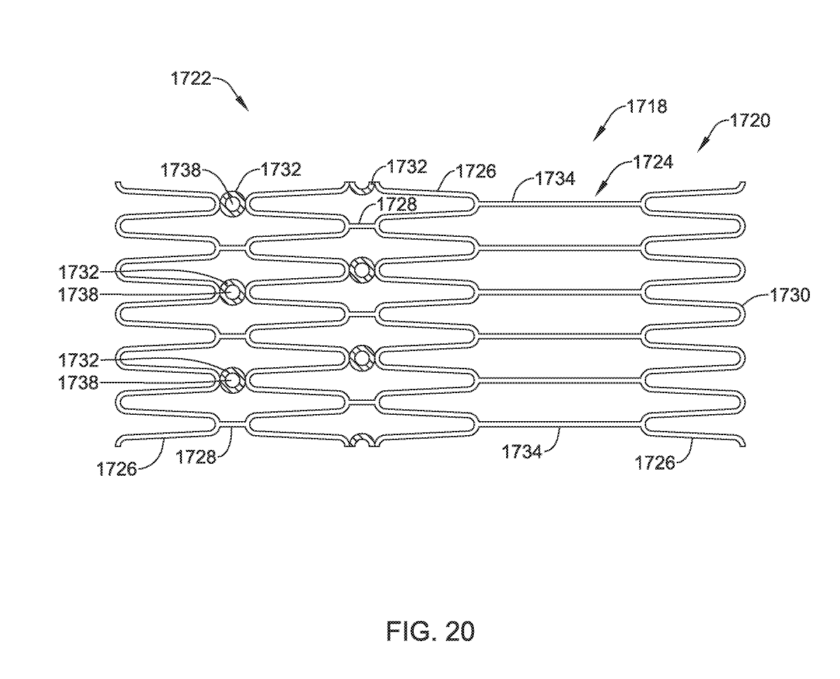

FIG. 20 illustrates another illustrative expandable basket 1718 cut open and flattened for clarity of illustration. It is contemplated that the expandable basket 1718 may be used in combination with any of the modulation systems disclosed herein. In some embodiments, the expandable basket 1718 may be formed from a conductive material covered with an insulating or semi-insulating coating 1730. Portions of the coating 1730 may be removed or omitted to form enlarged electrode pads or electrically conductive regions 1732. As shown in FIG. 20, electrically conductive regions 1732 may be formed as the electrically conductive regions 1732 may be formed as generally circular or annular rings between adjacent struts 1726. In some instances, the electrically conductive regions 1732 may be alternately formed with insulated connectors 1728 disposed therebetween. The electrically conductive regions 1732 may be disposed about a circumference of the basket 1718 to generally provide ablative energy around the circumference of a vessel. It is contemplated that the electrically conductive regions 1732 may be circumferentially staggered with an adjacent row of electrically conductive regions 1732. While the expandable basket 1718 is illustrated as having six electrically conductive regions 1732, it is contemplated that the basket may include any number of conductive regions 1732 desired, such as, but not limited to, one, two, three, four, or more.

It is contemplated that the outer surface of the basket 1718 (e.g. the surface capable of contacting a vessel wall) may be entirely coated with insulation 1730 while the electrically conductive regions 1732 are formed on an inner surface of the basket 1718. This may position the electrically conductive regions 1732 a short distance from the vessel wall when the basket 1718 is deployed. However, this is not required. It is contemplated that the electrically conductive regions may be positioned on any surface of the basket 1718 desired. In some embodiments, the electrically conductive regions 1732 may include one or more holes or openings 1738 disposed generally in the center of the circular ring. However, it is contemplated that openings 1738 take any shape desired such as, but not limited, longitudinal slots, or other geometric configurations. In some instances, openings 1738 in the enlarged electrically conductive regions 1732 may provide additional access for electrical current to flow from the electrically conductive regions 1732 to the target tissue.

As shown in FIG. 20, the proximal end region 1720 and the distal end region 1722 may include a one or more interconnected struts 1726 forming a generally cell-like pattern in combination with connectors 1728. It is contemplated that any number of struts 1726 and/or connectors 1728 may be used to form the basket 1718. In some instances, the proximal end region 1720 and the distal end region 1722 may be interconnected by an optional bridge segment 1724 including any number of generally longitudinally extending struts 1734. It is contemplated that in some instances, the bridge segment 1724 may be omitted. In such an instance, the electrically conductive regions 1732 may be positioned such that they are on a portion of the distal end region 1722 of the basket 1718 that is fully expanded when the basket 1718 is in the expanded configuration.

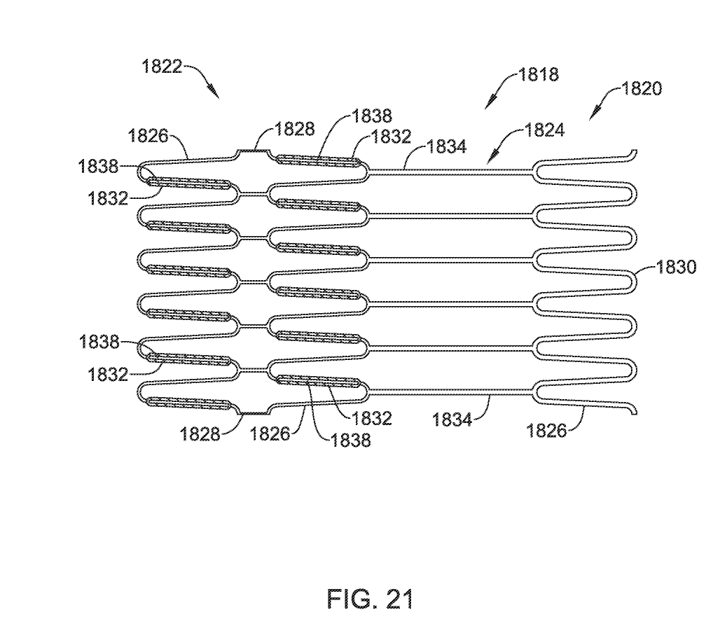

FIG. 21 illustrates another illustrative expandable basket 1818 cut open and flattened for clarity of illustration. It is contemplated that the expandable basket 1818 may be used in combination with any of the modulation systems disclosed herein. In some embodiments, the expandable basket 1818 may be formed from a conductive material covered with an insulating or semi-insulating coating 1830. Portions of the coating 1830 may be removed or omitted to form enlarged electrode pads or electrically conductive regions 1832. As shown in FIG. 21, electrically conductive regions 1832 may be formed as generally enlarged regions of the struts 1826. The electrically conductive regions 1832 may be separated by insulated regions of the struts 1826. The electrically conductive regions 1832 may be disposed about a circumference of the basket 1818 to generally provide ablative energy around the circumference of a vessel. It is contemplated that the electrically conductive regions 1832 on a strut 1826 may be circumferentially staggered with the electrically conductive regions 1832 on an adjacent strut 1826. While the expandable basket 1818 is illustrated as having twelve electrically conductive regions 1832, it is contemplated that the basket may include any number of conductive regions 1832 desired, such as, but not limited to, one, two, three, four, or more. It is further contemplated that the electrically conductive regions 1832 may be on the outside, inside, or side surfaces, or any combination thereof, of the struts 1826 or connectors 1828 as desired.

It is contemplated that the outer surface of the basket 1818 (e.g. the surface capable of contacting a vessel wall) may be entirely coated with insulation 1830 while the electrically conductive regions 1832 are formed on an inner surface of the basket 1818. This may position the electrically conductive regions 1832 a short distance from the vessel wall when the basket 1818 is deployed. However, this is not required. It is contemplated that the electrically conductive regions may be positioned on any surface of the basket 1818 desired. In some embodiments, the electrically conductive regions 1832 may include one or more fenestrations or openings 1838 disposed generally in the center of the oblong electrically conductive regions 1832. However, it is contemplated that openings 1838 take any shape desired such as, but not limited, longitudinal slots, holes, or other geometric configurations. In some instances, openings 1838 in the enlarged electrically conductive regions 1832 may provide additional access for electrical current to flow from the electrically conductive regions 1832 to the target tissue.

As shown in FIG. 21, the proximal end region 1820 and the distal end region 1822 may include a one or more interconnected struts 1826 forming a generally cell-like pattern in combination with connectors 1828. It is contemplated that any number of struts 1826 and/or connectors 1828 may be used to form the basket 1818. In some instances, the proximal end region 1820 and the distal end region 1822 may be interconnected by an optional bridge segment 1824 including any number of generally longitudinally extending struts 1834. It is contemplated that in some instances, the bridge segment 1824 may be omitted. In such an instance, the electrically conductive regions 1832 may be positioned such that they are on a portion of the distal end region 1822 of the basket 1818 that is fully expanded when the basket 1818 is in the expanded configuration.

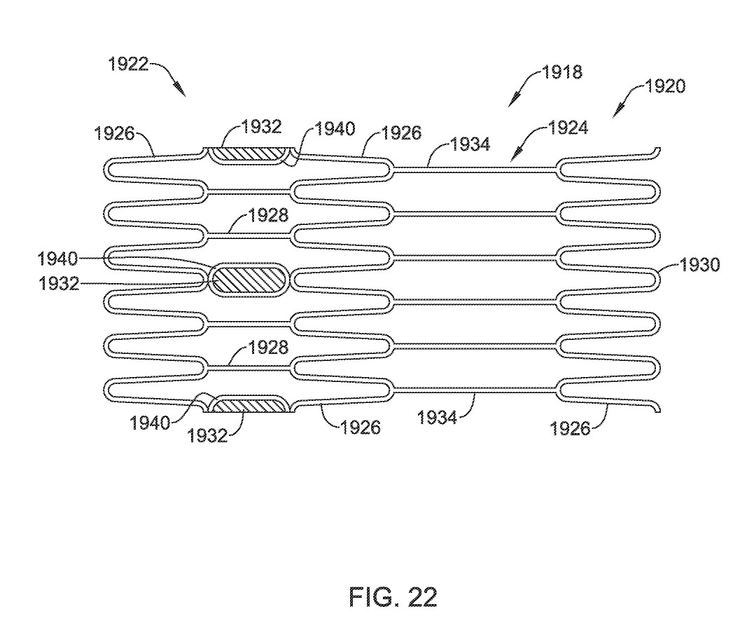

FIG. 22 illustrates another illustrative expandable basket 1918 cut open and flattened for clarity of illustration. It is contemplated that the expandable basket 1918 may be used in combination with any of the modulation systems disclosed herein. In some embodiments, the expandable basket 1918 may be formed from a conductive material covered with an insulating or semi-insulating coating 1930. Portions of the coating 1930 may be removed or omitted to form enlarged electrode pads or electrically conductive regions 1932. In some embodiments, the electrically conductive regions 1932 may be surrounded by a perimeter of insulation 1940. As shown in FIG. 22, the electrically conductive regions 1932 may be formed as generally oblong regions surrounded by an insulating edge 1940. The electrically conductive regions 1932 may be positioned between adjacent struts 1926. In some instances, the electrically conductive regions 1932 may be alternately formed with insulated connectors 1928 disposed therebetween. The electrically conductive regions 1932 may be disposed about a circumference of the basket 1918 to generally provide ablative energy around the circumference of a vessel. While the expandable basket 1918 is illustrated as having two electrically conductive regions 1932, it is contemplated that the basket may include any number of conductive regions 1932 desired, such as, but not limited to, one, two, three, four, or more.

It is contemplated that the outer surface of the basket 1918 (e.g. the surface capable of contacting a vessel wall) may be entirely coated with insulation 1930 while the electrically conductive regions 1932 may be formed on an inner surface of the basket 1918. This may position the electrically conductive regions 1932 a short distance from the vessel wall when the basket 1918 is deployed. The electrically conductive regions 1932 may be separated from the vessel wall by a distance that depends on the thickness of the insulation 1930 on the outside of the basket 1918 as well as the thickness and/or width of the insulating edge 1940. This separation distance may affect the current density in the adjacent wall and deeper tissues. The width of insulating edge 1940 may be chosen to be narrower if more concentrated, shallow heating is desired or wider if deeper heating that spares the artery wall is desired. Some electrically conductive regions 1932 can have a wider insulating edge 1940, while others have a narrower insulating edge 1940, to deliver more RF energy and more heating to some areas than to others. However, it is contemplated that the electrically conductive regions 1932 may be positioned on any surface of the basket 1918 desired. For example, electrically conductive regions 1932 may be positioned on an outer surface of the basket 1918. The perimeter of insulation 1940 may maintain the electrically conductive regions 1932 a short distance from the vessel wall. It is contemplated that the insulating edge 1940 may be uniform or can vary in width to preferentially heat some tissue regions more than others. For example, the width of the insulating edge 1940 can vary along the perimeter of a single electrically conductive region 1932 or it can vary between individual electrically conductive regions 1932 or a combination thereof.

As shown in FIG. 22, the proximal end region 1920 and the distal end region 1922 may include a plurality of interconnected struts 1926 forming a generally cell-like pattern in combination with connectors 1928. In some instances, the proximal end region 1920 and the distal end region 1922 may be interconnected by an optional bridge segment 1924 including any number of generally longitudinally extending struts 1934. It is contemplated that in some instances, the bridge segment 1924 may be omitted. In such an instance, the electrically conductive regions 1932 may be positioned such that they are on a portion of the distal end region 1922 of the basket 1918 that is fully expanded when the basket 1918 is in the expanded configuration. It is further contemplated that the electrically conductive regions 1932 may be on the outside, inside, or side surfaces, or any combination thereof, of the struts 1926 or connectors 1928 as desired.

It is contemplated that various basket 1918 geometries may be utilized. For example, the basket 1918 can include more or fewer interconnecting struts 1926, connectors 1928, etc. In some instances, the basket geometry can be chosen to obtain greater or lesser flexibility. Baskets with more rows or rings of struts 1926 can be used to spread out the heated areas more, at the expense of requiring a longer landing zone in the artery, and having a longer stiff section, especially during introduction and positioning. It is further contemplated that various patterns of insulated and non-insulated areas can be utilized to achieve the desired geometry of ablation. For example, the depth, volume, and temperature required for the treatment of a target tissue zone may affect the electrode (electrically conductive region) configuration that is required. The size and positioning of electrically conductive regions 1932 can be chosen to produce a more even heated zone, for example, such as by using a greater electrode area of non-wall-contact electrode(s) and a lesser area of wall contact electrode(s). Alternatively, an uneven or asymmetric heating zone can be obtained if desired.

The electrically conductive regions 1932 can be arranged so that they are reliably positioned as desired in the vessel when the distal end region 1922 of the basket 1918 expands. The configuration of the electrically conductive regions or electrodes 1932 may be chosen to obtain the desired heating of a volume of tissue. In some instances, the enlarged surface area of the electrically conductive regions 1932 relative to the other portions of the basket 1918 may provide room for insulation areas 1940 while still leaving sufficient electrode area. Wall-contact electrodes 1932 may provide more concentrated heating of nearby tissues and non-wall-contact electrodes 1932 may provide less concentrated heating of deeper tissues. Electrically conductive regions 1932 can be arranged in single or multiple contiguous areas, contiguous strips, circumferential rings, helical lines, or discrete spots, positioned closer or farther from each other and with various surface areas along the inside and/or outside of the basket 1918 to shape the heated zone as needed. Repeated patterns or combinations of arrangements can be used. Greater heating can be provided towards one end, or toward the middle, or along one side, compared to other areas, for example. The edge insulation 1940 thickness and/or width can be varied in combination with varying the number, size, and/or geometry (including fenestrations) of the electrically conductive regions 1932 to obtain the desired heating pattern.

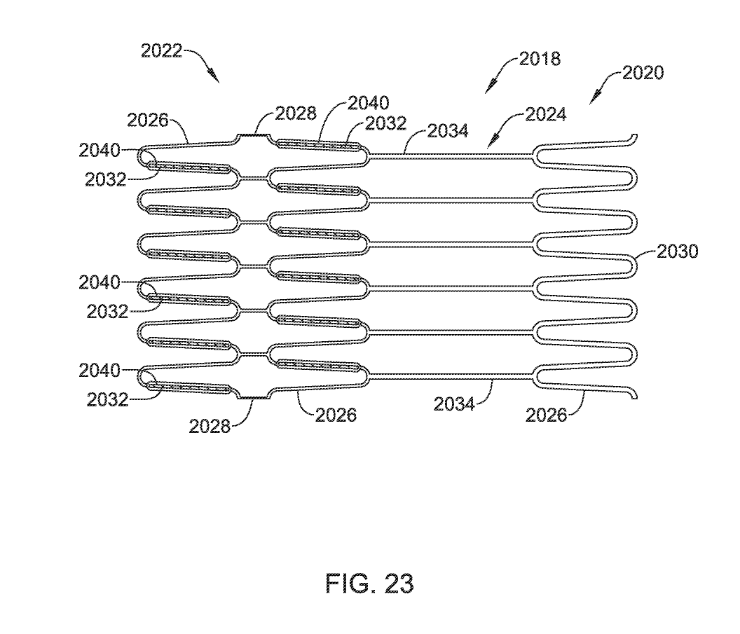

FIG. 23 illustrates another illustrative expandable basket 2018 cut open and flattened for clarity of illustration. It is contemplated that the expandable basket 2018 may be used in combination with any of the modulation systems disclosed herein. In some embodiments, the expandable basket 2018 may be formed from a conductive material covered with an insulating or semi-insulating coating 2030. Portions of the coating 2030 may be removed or omitted to form enlarged electrode pads or electrically conductive regions 2032. As shown in FIG. 23, electrically conductive regions 2032 may be formed as generally enlarged regions of the struts 2026. The electrically conductive regions 2032 may be separated by insulated regions of the struts 2026. The electrically conductive regions 2032 may be disposed about a circumference of the basket 2018 to generally provide ablative energy around the circumference of a vessel. It is contemplated that the electrically conductive regions 2032 on a strut 2026 may be circumferentially staggered with the electrically conductive regions 2032 on an adjacent strut 2026. While the expandable basket 2018 is illustrated as having twelve electrically conductive regions 2032, it is contemplated that the basket may include any number of conductive regions 2032 desired, such as, but not limited to, one, two, three, four, or more. In some instances, the electrically conductive regions 2032 may be surrounded by an insulating edge 2040. It is further contemplated that the electrically conductive regions 2032 may be on the outside, inside, or side surfaces, or any combination thereof, of the struts 2026 or connectors 2028 as desired.

It is contemplated that the outer surface of the basket 2018 (e.g. the surface capable of contacting a vessel wall) may be entirely coated with insulation 2030 while the electrically conductive regions 2032 are formed on an inner surface of the basket 2018. This may position the electrically conductive regions 2032 a short distance from the vessel wall when the basket 2018 is deployed. The electrically conductive regions 2032 may be separated from the vessel wall by a distance that depends on the thickness of the insulation 2030 on the outside of the basket 2018 as well as the thickness or width of the insulating edge 2040. This separation distance may affect the current density in the adjacent wall and deeper tissues. The width of the insulating edge 2040 may be chosen to be narrower if more concentrated, shallow heating is desired or wider if deeper heating that spares the artery wall is desired. However, it is contemplated that the electrically conductive regions may be positioned on any surface of the basket 2018 desired.

As shown in FIG. 23, the proximal end region 2020 and the distal end region 2022 may include a one or more interconnected struts 2026 forming a generally cell-like pattern in combination with connectors 2028. It is contemplated that any number of struts 2026 and/or connectors 2028 may be used to form the basket 2018. In some instances, the proximal end region 2020 and the distal end region 2022 may be interconnected by an optional bridge segment 2024 including any number of generally longitudinally extending struts 2034. It is contemplated that in some instances, the bridge segment 2024 may be omitted. In such an instance, the electrically conductive regions 2032 may be positioned such that they are on a portion of the distal end region 2022 of the basket 2018 that is fully expanded when the basket 2018 is in the expanded configuration.

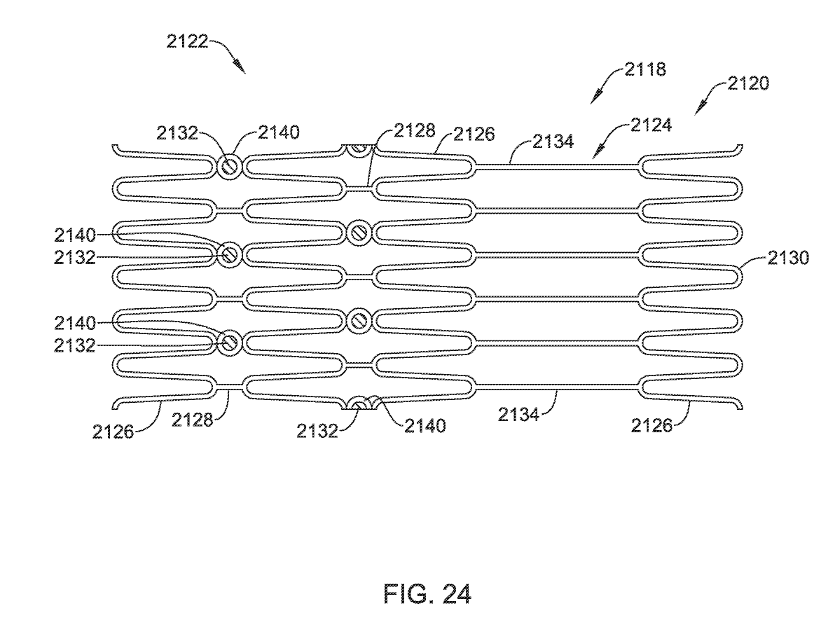

FIG. 24 illustrates another illustrative expandable basket 2118 cut open and flattened for clarity of illustration. It is contemplated that the expandable basket 2118 may be used in combination with any of the modulation systems disclosed herein. In some embodiments, the expandable basket 2118 may be formed from a conductive material covered with an insulating or semi-insulating coating 2130. Portions of the coating 2130 may be removed or omitted to form enlarged electrode pads or electrically conductive regions 2132. In some instances, electrically conductive regions 2132 may be formed as generally circular discs between adjacent struts 2126. In some instances, the electrically conductive regions 2132 may be alternately formed with insulated connectors 2128 disposed therebetween. The electrically conductive regions 2132 may be disposed about a circumference of the basket 2118 to generally provide ablative energy around the circumference of a vessel. It is contemplated that the electrically conductive regions 2132 may be circumferentially staggered with an adjacent row of electrically conductive regions 2132. While the expandable basket 2118 is illustrated as having six electrically conductive regions 2132, it is contemplated that the basket may include any number of conductive regions 2132 desired, such as, but not limited to, one, two, three, four, or more. In some instances, the electrically conductive regions 2132 may be surrounded by an insulating edge 2140. It is further contemplated that the electrically conductive regions 2132 may be on the outside, inside, or side surfaces, or any combination thereof, of the struts 2126 or connectors 2128 as desired.

It is contemplated that the outer surface of the basket 2118 (e.g. the surface capable of contacting a vessel wall) may be entirely coated with insulation 2130 while the electrically conductive regions 2132 are formed on an inner surface of the basket 2118. This may position the electrically conductive regions 2132 a short distance from the vessel wall when the basket 2118 is deployed. The electrically conductive regions 2132 may be separated from the vessel wall by a distance that depends on the thickness of the insulation 2130 on the outside of the basket 2118 as well as the thickness or width of the insulating edge 2140. This separation distance may affect the current density in the adjacent wall and deeper tissues. The width of the insulating edge 2140 may be chosen to be narrower if more concentrated, shallow heating is desired or wider if deeper heating that spares the artery wall is desired. However, it is contemplated that the electrically conductive regions may be positioned on any surface of the basket 2118 desired.

As shown in FIG. 24, the proximal end region 2120 and the distal end region 2122 may include a one or more interconnected struts 2126 forming a generally cell-like pattern in combination with connectors 2128. It is contemplated that any number of struts 2126 and/or connectors 2128 may be used to form the basket 2118. In some instances, the proximal end region 2120 and the distal end region 2122 may be interconnected by an optional bridge segment 2124 including any number of generally longitudinally extending struts 2134. It is contemplated that in some instances, the bridge segment 2124 may be omitted. In such an instance, the electrically conductive regions 2132 may be positioned such that they are on a portion of the distal end region 2122 of the basket 2118 that is fully expanded when the basket 2118 is in the expanded configuration.

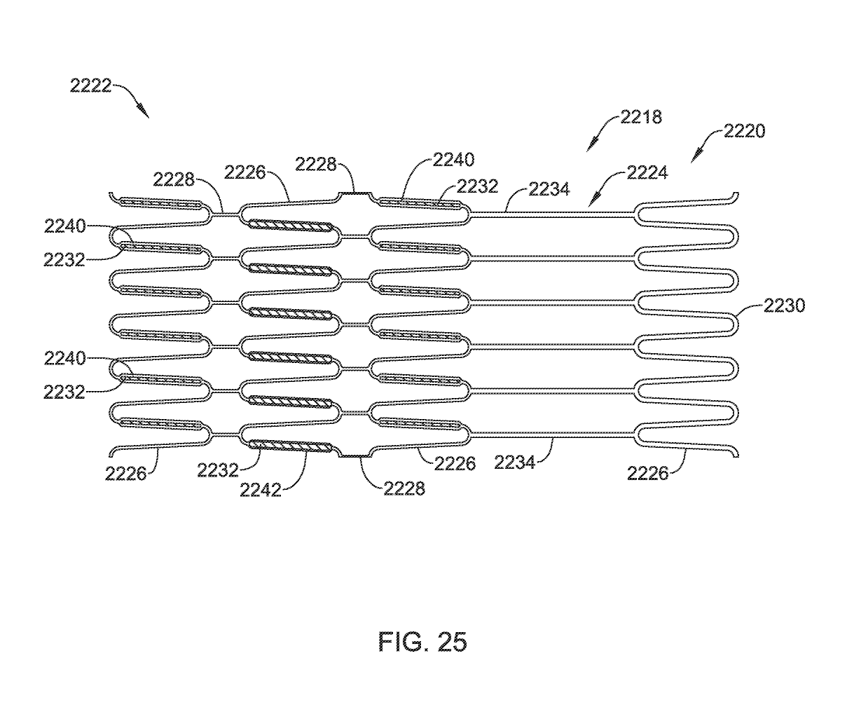

FIG. 25 illustrates another illustrative expandable basket 2218 cut open and flattened for clarity of illustration. It is contemplated that the expandable basket 2218 may be used in combination with any of the modulation systems disclosed herein. In some embodiments, the expandable basket 2218 may be formed from a conductive material covered with an insulating or semi-insulating coating 2230. Portions of the coating 2230 may be removed or omitted to form enlarged electrode pads or electrically conductive regions 2232. As shown in FIG. 25, the electrically conductive regions 2232 may be formed as generally enlarged regions of the struts 2226. The electrically conductive regions 2232 may be separated by insulated regions of the struts 2226. The electrically conductive regions 2232 may be disposed about a circumference of the basket 2218 to generally provide ablative energy around the circumference of a vessel. It is contemplated that the electrically conductive regions 2232 on a strut 2226 may be circumferentially staggered with the electrically conductive regions 2232 on an adjacent strut 2226. While the expandable basket 2218 is illustrated as having eighteen electrically conductive regions 2232, it is contemplated that the basket 2218 may include any number of conductive regions 2232 desired, such as, but not limited to, one, two, three, four, or more. In some instances, the electrically conductive regions 2232 may be surrounded by an insulating edge 2240, 2242. It is further contemplated that the electrically conductive regions 2232 may be on the outside, inside, or side surfaces, or any combination thereof, of the struts 2226 or connectors 2228 as desired.

It is contemplated that the outer surface of the basket 2218 (e.g. the surface capable of contacting a vessel wall) may be entirely coated with insulation 2230 while the electrically conductive regions 2232 are formed on an inner surface of the basket 2218. This may position the electrically conductive regions 2232 a short distance from the vessel wall when the basket 2218 is deployed. The electrically conductive regions 2232 may be separated from the vessel wall by a distance that depends on the thickness of the insulation 2230 on the outside of the basket 2218 as well as the thickness or width of the insulating edge 2240, 2242. This separation distance may affect the current density in the adjacent wall and deeper tissues. The width of the insulating edge 2240, 2242 may be chosen to be narrower if more concentrated, shallow heating is desired or wider if deeper heating that spares the artery wall is desired. In some embodiments, some electrically conductive regions 2232 may have a first wider insulating edge 2240 while other electrically conductive regions 2232 may have a second narrower insulating edge 2242. It is contemplated that the wider edge insulation 2240 may be present on certain struts 2226 while the narrower edge insulation 2242 may be present on other struts 2226. However, the pattern, or lack of pattern, of the width of the edge insulation 2240, 2242 may be selected to achieve the desired heating pattern. It is further contemplated that the electrically conductive regions may be positioned on any surface of the basket 2218 desired.

As shown in FIG. 25, the proximal end region 2220 and the distal end region 2222 may include a one or more interconnected struts 2226 forming a generally cell-like pattern in combination with connectors 2228. It is contemplated that any number of struts 2226 and/or connectors 2228 may be used to form the basket 2218. In some instances, the proximal end region 2220 and the distal end region 2222 may be interconnected by an optional bridge segment 2224 including any number of generally longitudinally extending struts 2234. It is contemplated that in some instances, the bridge segment 2224 may be omitted. In such an instance, the electrically conductive regions 2232 may be positioned such that they are on a portion of the distal end region 2222 of the basket 2218 that is fully expanded when the basket 2218 is in the expanded configuration.

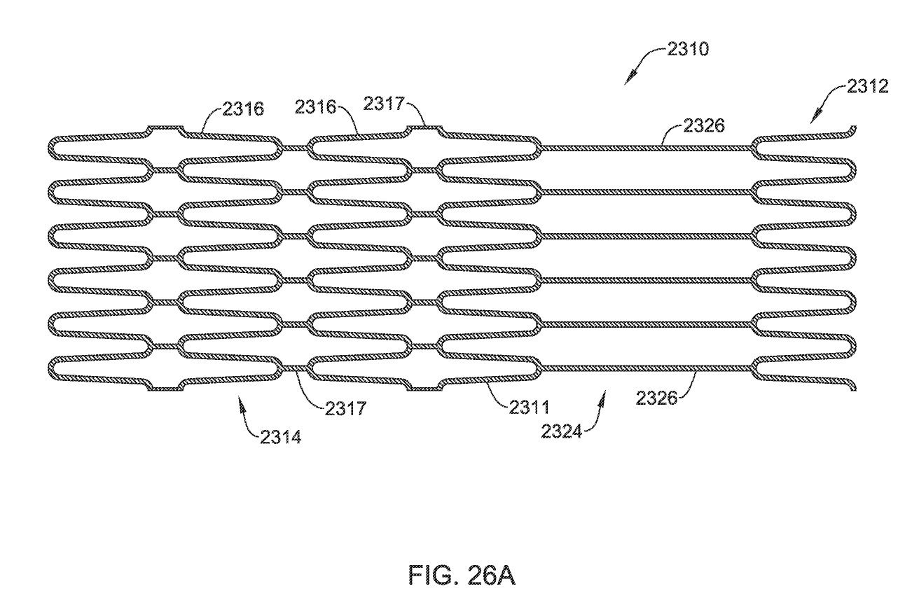

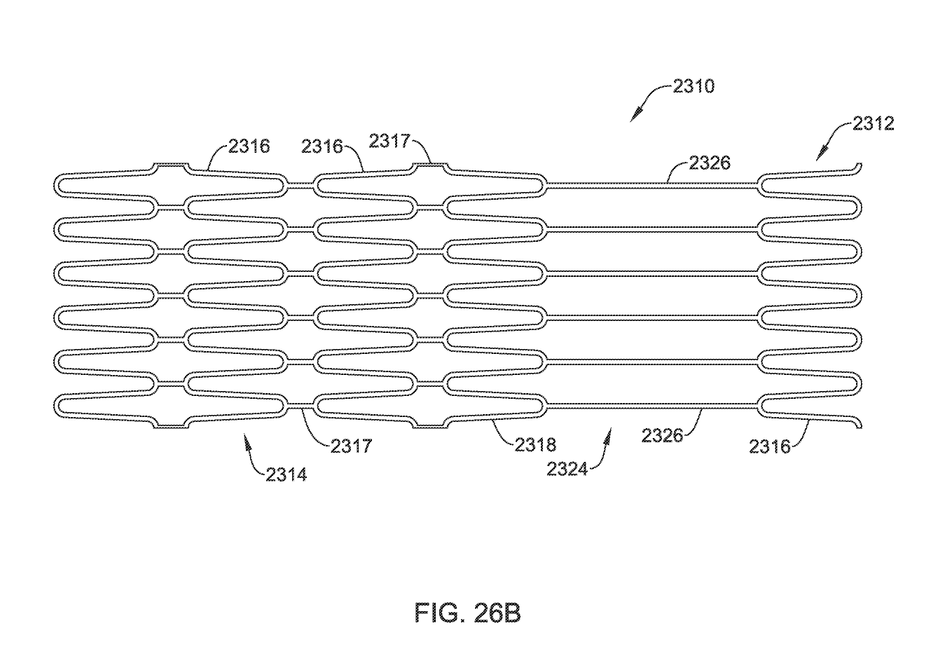

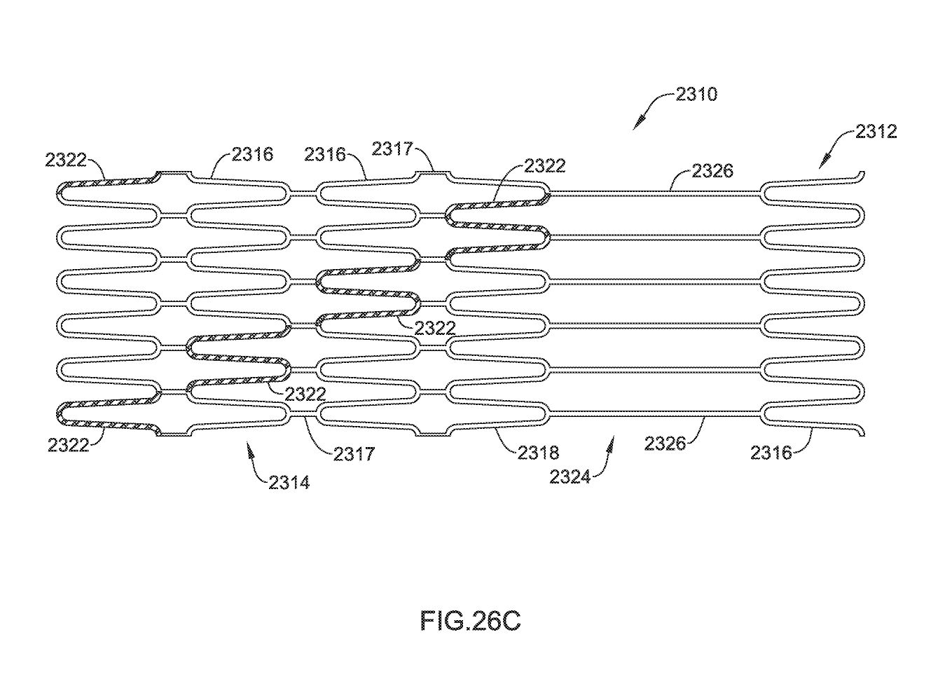

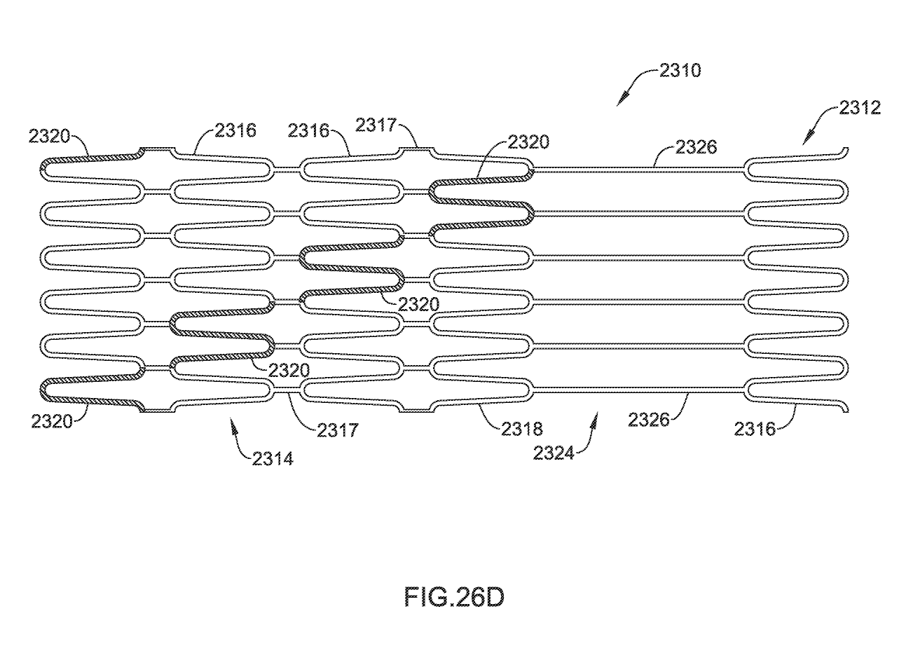

FIGS. 26A-26D illustrate an illustrative method for forming an illustrative expandable basket having electrically conductive regions. FIG. 26A illustrates an illustrative expandable basket 2310 cut open and flattened for clarity of illustration. The expandable basket 2310 may have a proximal end region 2312, a distal end region 2314, and an optional bridge segment 2324 disposed therebetween. In some embodiments, the expandable basket 2310 may be formed from a plurality of interconnected circumferentially extending struts 2316. The struts 2316 may be connected by one or more connectors 2317. In some instances, the proximal end region 2312 and the distal end region 2314 may be interconnected by an optional bridge segment 2324 including any number of generally longitudinally extending struts 2326. It is contemplated that in some instances, the bridge segment 2324 may be omitted.

In some instances, the basket 910 may be formed from a metal, or other conductive material. However, it is contemplated that the basket 2310 may be formed of a non-conductive material. In such an instance, one or more conductive traces (not explicitly shown may be provided within or on a surface of the basket 2310 to conduct RF energy to the electrically conductive regions. It is further contemplated that when a non-conductive basket 2310 is utilized electrically conductive regions may be provided as discrete electrically conductive elements affixed to a surface of the basket 2310.

In some embodiments, the expandable basket 2310 may be cut from a generally tubular member to form a basket 2310 having the desired frame 2311 geometry. It is contemplated that the tubular member may be cut using chemical, laser, electron discharge machining (EDM), or other known technologies. It is contemplated that a various heat treatment approaches can be used with particular metals to impart temper, flexibility, self-expansion, self-collapsing, or shape memory attributes using known technologies.

Once the frame 2311 has been formed, the basket 2310 may be coated, entirely or partially, by a coating 2318 such as, but not limited to parylene, fluoropolymers, porous polymers, ceramics, etc., as shown in FIG. 26B. Typical coating processes may be used to apply the coating, such as, but not limited to, dip coating, spray coating, etc. While FIG. 26B illustrates the entire basket 2310 as coated with a coating 2318, it is contemplated that only a portion of the basket 2310 may be coated. For example, the coating 2318 may be selectively applied to portions of the basket 2310 using finely controlled application processing, such as, but not limited to inkjet printing technology. When the coating 2318 is selectively applied, electrically conductive regions 2320 (see FIG. 26D) may be left uncoated intentionally. It is further contemplated that portions of the basket 2310 may be selectively masked (not explicitly shown) prior to coating the basket 2310 with coating 2318 to create electrically conductive regions 2320. For example, modified chemical adhesives (carbonate urethanes, Loctite® 3106™ light cure adhesive) or thermally processed masks (polyethylene, thermoplastic polyurethane, or other “hotmelt” type materials applied at the locations to be masked). Alternatively, the mask may be selectively applied to portions of the stent using finely controlled application processing, such as inkjet printing technology. After coating, the masking is removed (using physical approaches such as “picking off” or “scraping off” the mask, or by thermal approaches such as melting the mask material, or other approaches.

Regardless of whether a masking material has been used or not, once the coating 2318 has been applied, the coating 2318 may be selectively removed from the basket 2310 either at the masked location or an unmasked location. The coating 2318 may be scored 2322 as shown in FIG. 26C to facilitate removal of the coating 2318 at the desired location. It is contemplated that the coating 2318 may be selectively removed using physical approaches such as scoring or cutting and “picking off” or “scraping off” the mask, or by thermal approaches such as melting the mask material, laser or other energy approach, or other approaches. Selectively removing the coating 2318 will expose regions of the electrically conductive frame 2311 thereby forming electrically conductive regions 2320, as shown in FIG. 26D. It is contemplated that the electrically conductive regions 2320 may be formed about the expandable basket 2310 in any manner desired to provide the desired ablation pattern. It is further contemplated that the electrically conductive regions 2320 may be on the outside, inside, or side surfaces, or any combination thereof, of the struts 2316 or connectors 2317 as desired.

Devices 100, 1000 or any of the baskets disclosed herein may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of devices 100, 1000 or any of the baskets disclosed herein may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of devices 100, 1000 or any of the baskets disclosed herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of devices 100, 1000 or any of the baskets disclosed herein to achieve the same result.

In some embodiments, a degree of Magnetic Resonance Imaging (MRI) compatibility is imparted into devices 100, 1000 or any of the baskets disclosed herein. For example, devices 100, 1000 or any of the baskets disclosed herein, or portions thereof, may be made of a material that does not substantially distort the image and create substantial artifacts (i.e., gaps in the image). Certain ferromagnetic materials, for example, may not be suitable because they may create artifacts in an MRI image. Devices 100, 1000 or any of the baskets disclosed herein or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers for devices 100, 1000 or any of the baskets disclosed herein may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Those skilled in the art will recognize that the present disclosed subject matter may be manifested in a variety of forms other than the specific embodiments described and contemplated herein. Accordingly, departure in form and detail may be made without departing from the scope and spirit of the present disclosure as described in the appended claims.

What is claimed is:

1. An intravascular nerve modulation device, comprising:
an elongate shaft having a proximal end region and a distal end region;
an expandable basket having a proximal end region and a distal end region, the proximal end region of the expandable basket affixed to the elongate shaft adjacent to the distal end region of the elongate shaft;
a coating disposed over at least a portion of the expandable basket; and
one or more electrically conductive regions disposed along a portion of the distal end region of the expandable basket.

2. The nerve modulation device of claim 1, wherein the expandable basket comprises an electrically conductive material.

3. The nerve modulation device of claim 2, wherein one or more electrically conductive regions are regions of the expandable basket free of the coating.

4. The nerve modulation device claim 1, wherein the expandable basket comprises an electrically non-conductive material.

5. The nerve modulation system of claim 4, wherein the one or more electrically conductive regions are discrete electrically conductive elements affixed to the expandable basket.

6. The nerve modulation device of claim 1, wherein the one or more electrically conductive regions comprise a plurality of electrically conductive regions.

7. The nerve modulation device of claim 6, wherein the plurality of electrically conductive regions are disposed about the expandable basket in a helical pattern.

8. The nerve modulation device of claim 6, wherein the plurality of electrically conductive regions are spaced about a circumference of the expandable basket.

9. The nerve modulation device of claim 1, wherein the expandable basket comprises a plurality of interconnected struts extend circumferentially in a serpentine manner.

10. The nerve modulation device of claim 9, wherein the struts include a plurality of relatively straight portions and a plurality of curved portions joining adjacent straight portions.

11. The nerve modulation device of claim 10, wherein the curved portions have a cross-sectional area smaller than a cross-sectional are of the relatively straight portions.

12. An intravascular nerve modulation device, comprising:
an elongate shaft having a proximal end region and a distal end region;
a self-expanding basket including a plurality of interconnected struts forming a network of cells, the basket having a proximal end region and a distal end region, the proximal end region of the basket affixed to the elongate shaft adjacent to the distal end region of the elongate shaft;
an insulating coating disposed over a least a portion of the basket; and
one or more electrically conductive regions disposed along a portion of the distal end region of the basket.

13. The nerve modulation device of claim 12, wherein the basket comprises an electrically conductive material.

14. The nerve modulation device of claim 13, wherein the one or more electrically conductive regions are regions of the basket free of the insulating coating.

15. A method for manufacturing a medical device, the method comprising:
coating an expandable basket formed of a conductive material with an insulating or semi-insulating material; and
removing selective portions of the insulating or semi-insulating material to define electrically conductive regions.

16. The method of claim 15, wherein coating the expandable basket comprises dip coating the basket.

17. The method of claim 16, wherein coating the expandable basket comprises spray coating the basket.

18. The method of claim 16, further comprising scoring the insulating material at the selective portions prior to removing said selective portions of the insulating or semi-insulating material.

19. The method of claim 16, wherein prior to coating the expandable basket with the insulating or semi-insulating material, the basket is selectively masked.

20. The method of claim 19, wherein removing selective portions of the insulating or semi-insulating material comprises removing a masking material.
